(19) United States
(12) Patent Application Publication
(10) Pub. No.: US 2025/0122193 A1
(43) Pub. Date: Apr. 17, 2025

(54) NOVEL PYRIDOCARBAZOLIUM COMPOUNDS AND MEDICAL USES THEREOF

(71) Applicant: MyCural Therapeutics, Uppsala (SE)

(72) Inventors: Karin Fawkner, Uppsala (SE); Lars Johansson, Solna (SE); Lars-Gunnar Olof Larsson, Stockholm (SE); Alina Andrea Viktoria Castell, Stockholm (SE)

(21) Appl. No.: 18/721,989

(22) PCT Filed: Dec. 22, 2022
(86) PCT No.: PCT/GB2022/053376

§ 371 Date: Jun. 20, 2024

(30) Foreign Application Priority Data
Dec. 22, 2021 (GB) 2118752.1
Apr. 12, 2022 (GB) 2205404.3

(51) Int. Cl. C07D 471/04; A61K 31/4745; A61K 45/06; A61P 35/00; A61P 35/02

(52) U.S. Cl. CPC C07D 471/04; A61K 31/4745; A61K 45/06; A61P 35/00; A61P 35/02

(57) ABSTRACT

There is provided herein compounds of formula I: (I) and pharmaceutically-acceptable salts and/or detectably-labelled derivatives thereof, wherein R^{A}, R^{B}, X, Y, n and m have meanings as provided in the description, together with formulations and products comprising the same. There is also provided the use of such compounds, formulations and products in the treatment of cancers, such as cancers characterised by increased MYC activity.

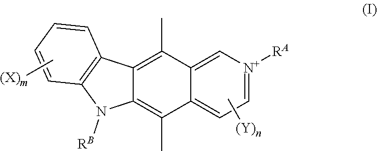

### FIELD OF THE INVENTION

The present invention relates to novel compounds, compositions comprising such compounds, and the use of such compounds and compositions in medicine. In particular, the present invention relates to the use of such compounds and compositions in methods for the treatment of cancers, particularly cancers characterised by increased activity of the MYC pathway, which treatment is thought to occur through specific and potent inhibition of MYC:MAX interaction.

### BACKGROUND OF THE INVENTION

The listing or discussion of an apparently prior-published document in this specification should not necessarily be taken as an acknowledgement that the document is part of the state of the art or is common general knowledge.

The MYC family of oncogenes, consisting of MYC, MYCN and MYCL (here collectively referred to as “MYC”), encodes basic helix-loop-helix leucine zipper (bHLHZip) transcription factors (Meyer, N. & Penn, L. Z., Nat Rev Cancer, 8, 976-990 (2008)). Through the HLHZip domain, MYC heterodimerizes with the bHLHZip protein MAX, which enables the MYC:MAX complex to bind E-box regulatory DNA elements throughout the genome, thereby controlling transcription of a large group of specific genes. The direct target gene products in turn influences global RNA and protein synthesis, thereby coordinating multiple fundamental cellular processes, including cell cycle progression, cell growth, apoptosis, senescence, metabolism and stem cell functions. In addition to MAX, MYC interacts with a plethora of other proteins carrying out different functions in gene regulation. Deregulation of expression of MYC family genes/proteins occurs in over half of all human tumors, and can be caused by chromosomal aberrations affecting the MYC loci, such as translocation and gene amplification, or can be due to oncogenic aberrations affecting upstream regulators of MYC. Abnormal MYC expression is often correlated with aggressive disease, resistance to therapy and poor prognosis, and MYC is therefore considered as one of the most important drivers of tumor development. Evidence from mouse models has shown that elimination of MYC using genetic tools often causes complete and irreversible tumor regression with well-tolerated and reversible side effects, suggesting that MYC would be a suitable target for cancer therapy (Soucek, L. et al., Nature, 455, 679-683 (2008)). However, so far there are no specific anti-MYC drugs available in the clinic.

Although MYC has previously been considered “undruggable”, a number of efforts have been made during recent years to target MYC. MYC expression is sensitive to BET bromodomain inhibitors and to protein translation inhibitors in certain cells. Alternatively, kinases regulating MYC activity and turnover, druggable key downstream MYC target gene products or synthetic lethal interactions involving MYC can be targeted (McKeown, M. R. & Bradner, J. E., Cold Spring Harb Perspect Med, 4, (2014)). However, these approaches are not specific for MYC, are context-dependent, and are presumably bound to fail eventually due to selection of alternative pathways regulating MYC expression and activity in tumor cells.

While these strategies all target MYC indirectly, several efforts to target the MYC protein directly have also been reported (Fletcher, S. & Prochownik, E. V., Biochim Biophys Acta 1849, 525-543 (2015)). Since MYC is strictly dependent on MAX for binding E-boxes, targeting MYC:MAX or MYC:MAX:DNA interactions are therefore a conceivable approach to target MYC activity (Blackwood, E. M. & Eisenman, R. N., Science 251, 1211-1217 (1991)). This suggests that the MYC:MAX heterodimer could be a potential drug target in vivo. However, targeting protein-protein interactions (PPI) is challenging due to presumed large, flat interactions surfaces lacking pockets amenable for small-molecule binding (Nero et al., 2014). In addition, the monomeric MYC bHLHZip domain is intrinsically disordered, and adopts an α-helical HLHZip fold upon dimerization with MAX (Metallo, 2010; Nair and Burley, 2003). Nevertheless, it has become clear that PPIs often involve “hot spots” engaging a small number, or cluster, of residues where most of the binding energy is localized, and therefore potentially druggable with small molecules (Nero, T. L., Morton, C. J., Holien, J. K., Wielens, J. & Parker, M. W., Nature reviews. Cancer, 14, 248-262 (2014); Zinzalla, G., and Thurston, D. E., Future Med Chem, 1, 65-93 (2009)).

During recent years there have been several reports of successful targeting of protein-protein interactions with small molecules, including Nutlin-3a (targeting p53:MDM2) (Vassilev, L. T. et al., Science, 303, 844-848 (2004)), BET inhibitors such as JQ1 (bromodomains:histones) (Filippakopoulos, P. et al., Nature, 468, 1067-1073 (2010)) and the BH3 mimetic compound Navitoclax/ABT-263 (BCL-2 family protein interactions) (Tse, C. et al., Cancer Res, 68, 3421-3428 (2008)). These compounds, or improved versions, are now in clinical trials, which have encouraged further research on PPIs as drug targets (Arkin, M. R., Tang, Y., and Wells, J. A., Chemistry & biology, 21, 1102-1114 (2014); Nero, T. L., Morton, C. J., Holien, J. K., Wielens, J. & Parker, M. W., Nature reviews. Cancer, 14, 248-262 (2014)).

Several research groups have attempted to find compounds targeting the MYC:MAX interaction by screening small-molecule libraries using for instance FRET, or fluorescence polarization in vitro, or by applying yeast-two-hybrid (Y2H) assays or cell-based interaction assays (Fletcher, S. & Prochownik, E. V., Biochim Biophys Acta 1849, 525-543 (2015), Duffy M J, O'Grady S, Tang M, & Crown J (2021) MYC as a target for cancer treatment. Cancer Treat Rev 94:102154). Another approach has been the design of peptidomimetic molecules targeting MYC:MAX PPIs based on the structures of the HLHZip region of MYC or MAX, or the Omomyc cell-penetrating mini-protein (Giorello, L. et al., Cancer Res, 58, 3654-3659 (1998), Beaulieu M E, et al. (2019) Intrinsic cell-penetrating activity propels Omomyc from proof of concept to viable anti-MYC therapy. Sci Transl Med 11(484)). As a result, a number of compounds have been identified that target the MYC:MAX or MYC:MAX:DNA interaction in vitro and in mammalian cells, and that inhibit MYC-driven tumor cell growth in cell cultures and to some extent in vivo (Fletcher, S. & Prochownik, E. V., Biochim Biophys Acta 1849, 525-543 (2015); McKeown, M. R. & Bradner, J. E., Cold Spring Harb Perspect Med, 4, (2014), Duffy M J, O'Grady S, Tang M, & Crown J (2021) MYC as a target for cancer treatment. Cancer Treat Rev 94:102154, Whitfield J R, Beaulieu M E, & Soucek L (2017) Strategies to Inhibit Myc and Their Clinical Applicability. Front Cell Dev Biol 5:10). However, none of these compounds have made their way for clinical studies.

Given this, there exists an urgent need to identify and develop new potent and selective direct MYC inhibitors suitable for in vivo applications, such as for the treatment of cancers.

WO 2019/145375 describes diazinyl diamino acridines and the use of such compounds, formulations and products in the treatment of cancers characterised by increased MYC activity.

### DETAILED DESCRIPTION OF THE INVENTION

It has now been found that certain pyridocarbazolium compounds have surprising properties which render such compounds useful in the treatment of cancers, particularly those cancers characterised by increased activity of the MYC pathway (i.e. increased MYC activity).

In particular, by using a mammalian cell-based MYC:MAX protein interaction screen, it has now surprisingly been found that these pyridocarbazolium compounds strongly interfere with MYC:MAX interaction in cells and in vitro. It has been found that these compounds are unexpectedly able to inhibit MYC-driven transcription by binding MYC directly in vitro, in turn inhibiting MYC-dependent tumor cell growth both in cell cultures and in a MYC-driven mouse xenograft tumor model, while sparing normal cells.

### Compounds of the Invention

In a first aspect of the invention, there is provided a compound of formula I

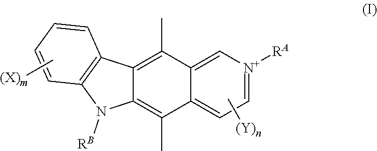

or a pharmaceutically-acceptable salt and/or detectably labelled derivative thereof, wherein:
R^{A} represents C₁₋₆ alkyl optionally substituted by one or more F, oxo or R^{A1};
R^{A1} represents —OR^{A2}, —N(R^{A3})R^{A4}, halo, CN, or phenyl optionally substituted by one or more halo;
R^{A2} represents H or C₁₋₃ alkyl optionally substituted with one or more F;
R^{A3} and R^{A4} each represent H or C₁₋₃ alkyl optionally substituted with one or more F,
or alternatively R^{A3} and R^{A4} may be taken together to form a C₄₋₅ alkylene optionally substituted with one or more F;
R^{B} represents H or C₁₋₃ alkyl optionally substituted with one or more F;
m represents 0, 1, 2, 3 or 4;
n represents 0, 1, 2 or 3;
each X independently represents —OR^{A5}, —N(R^{A6})R^{A7}, halo, —CN or C₁₋₃ alkyl optionally substituted with one or more F; and
R^{A5} represents H or C₁₋₃ alkyl optionally substituted with one or more F;
R^{A6} and R^{A7} each represent H or C₁₋₃ alkyl optionally substituted with one or more F; and
each Y represents methyl,
which compounds of formula I may be referred to herein as the “compounds of the invention”.

For the avoidance of doubt, the skilled person will understand that references herein to compounds of particular aspects of the invention (such as the first aspect of the invention, i.e. referring to compounds of formula I as defined in the first aspect of the invention) will include references to all embodiments and particular features thereof, which embodiments and particular features may be taken in combination to form further embodiments and features of the invention.

Unless indicated otherwise, all technical and scientific terms used herein will have their common meaning as understood by one of ordinary skill in the art to which this invention pertains.

For the avoidance of doubt, the compounds of the invention are cationic and therefore exist as salts in association with a suitable (e.g. pharmaceutically acceptable) anionic counter-ion. Particular counterions (anions) that may be mentioned include bromide (Br⁻), iodide (I⁻) and acetate (AcO⁻).

Thus, compounds of formula I may be referred to as being in the presence of a suitable anion, which anion alternatively may be depicted in formula I as A⁻.

Pharmaceutically acceptable salts include acid addition salts. Such salts may be formed by conventional means, for example by reaction of a free base form of a precursor of the compound of the invention with one or more equivalents of an appropriate acid, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalene-disulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

For the avoidance of doubt, compounds of the invention may exist as solids, and thus the scope of the invention includes all amorphous, crystalline and part crystalline forms thereof, and may also exist as oils. Where compounds of the invention exist in crystalline and part crystalline forms, such forms may include solvates, which are included in the scope of the invention.

For the avoidance of doubt, compounds of the invention may also exist in solution (i.e. in solution in a suitable solvent). For example, compounds of the invention may exist in aqueous solution, in which case compounds of the invention may exist in the form of hydrates thereof.

Compounds of the invention (and similarly, compounds excluded from the scope of the invention) may contain double bonds and, unless otherwise indicated, may thus exist as E (entgegen) and Z (zusammen) geometric isomers about each individual double bond. Unless otherwise specified, all such isomers and mixtures thereof are included within the scope of the invention (or within the relevant exclusion).

For the avoidance of doubt, where a bond is indicated by a wavy line, the skilled person will understand that the substituent bond by that bond may be present in the E or Z configuration.

Compounds of the invention may also exhibit tautomerism. All tautomeric forms and mixtures thereof are included within the scope of the invention (particularly those of sufficient stability to allow for isolation thereof).

Compounds of the invention may also contain one or more asymmetric carbon atoms and may therefore exhibit optical and/or diastereoisomerism (i.e. existing in enantiomeric or diastereomeric forms). Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired enantiomer or diastereoisomer may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution; for example, with a homochiral acid followed by separation of the diastereomeric derivatives by conventional means such as chromatography), or by reaction with an appropriate chiral reagent or chiral catalyst, all of which methods and processes may be performed under conditions known to the skilled person. Unless otherwise specified, all stereoisomers and mixtures thereof are included within the scope of the invention.

For the avoidance of doubt, the skilled person will understand that where a particular group is depicted herein as being bound to a ring system via a floating bond (i.e. a bond not shown as being bound to a particular atom within the ring), the relevant group may be bound to any suitable atom within the relevant ring system (i.e. the ring within which the floating bond terminates).

Unless otherwise specified, C_{1-z} alkyl groups (where z is the upper limit of the range) defined herein may be straight-chain or, when there is a sufficient number (i.e. a minimum of two or three, as appropriate) of carbon atoms, be branched-chain, and/or cyclic (so forming a C_{3-z} cycloalkyl group). When there is a sufficient number (i.e. a minimum of four) of carbon atoms, such groups may also be part cyclic (so forming a C_{4-z} partial cycloalkyl group). For example, cycloalkyl groups that may be mentioned include cyclopropyl, cyclopentyl and cyclohexyl. Similarly, part cyclic alkyl groups (which may also be referred to as “part cycloalkyl” groups) that may be mentioned include cyclopropylmethyl. When there is a sufficient number of carbon atoms, such groups may also be multicyclic (e.g. bicyclic or tricyclic) and/or spirocyclic. For the avoidance of doubt, particular alkyl groups that may be mentioned include straight chain (i.e. not branched and/or cyclic) alkyl groups.

For example, particular C₁₋₃ alkyl groups that may be mentioned include methyl, ethyl, isopropyl and n-propyl.

Unless otherwise specified, C_{2-z} alkenyl groups (where z is the upper limit of the range) defined herein may be straight-chain or, when there is a sufficient number (i.e. a minimum of three) of carbon atoms, be branched-chain, and/or cyclic (so forming a C_{4-z} cycloalkenyl group). When there is a sufficient number (i.e. a minimum of five) of carbon atoms, such groups may also be part cyclic. For example, part cyclic alkenyl groups (which may also be referred to as “part cycloalkenyl” groups) that may be mentioned include cyclopentenylmethyl and cyclohexenylmethyl. When there is a sufficient number of carbon atoms, such groups may also be multicyclic (e.g. bicyclic or tricyclic) or spirocyclic. For the avoidance of doubt, particular alkenyl groups that may be mentioned include straight chain (i.e. not branched and/or cyclic) alkenyl groups.

Unless otherwise specified, C_{2-z} alkynyl groups (where z is the upper limit of the range) defined herein may be straight-chain or, when there is a sufficient number (i.e. a minimum of four) of carbon atoms, be branched-chain. For the avoidance of doubt, particular alkynyl groups that may be mentioned include straight chain (i.e. not branched and/or cyclic) alkynyl groups.

For the avoidance of doubt, unless otherwise specified, groups referred to herein as “alkyl”, “alkenyl” and/or “alkynyl” will be taken as referring to the highest degree of unsaturation in a bond present in such groups. For example, such a group having a carbon-carbon double bond and, in the same group, a carbon-carbon triple bond will be referred to as “alkynyl”. Alternatively, it may be particularly specified that that such groups will comprise only the degree of unsaturation specified (i.e. in one or more bond therein, as appropriate; e.g. in in one bond therein).

For the avoidance of doubt, alkyl, alkenyl and alkynyl groups as described herein may also act as linker groups (i.e. groups joining two or more parts of the compound as described), in which case such groups may be referred to as “alkylene”, “alkenylene” and/or “alkynylene” groups, respectively.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulfur (e.g. oxygen, nitrogen and sulfur, such as oxygen and nitrogen).

As used herein, the term heterocyclyl may refer to non-aromatic monocyclic and polycyclic (e.g. bicyclic) heterocyclic groups (which groups may, where containing a sufficient number of atoms, also be bridged) in which at least one (e.g. one to four) of the atoms in the ring system is other than carbon (i.e. a heteroatom), and in which the total number of atoms in the ring system is between three and twelve (e.g. between five and ten, such as between three and eight; for example, forming a 5- or 6-membered heterocyclyl group). Further, such heterocyclyl groups may be saturated, forming a heterocycloalkyl, or unsaturated containing one or more carbon-carbon or, where possible, carbon-heteroatom or heteroatom-heteroatom double and/or triple bonds, forming for example a C_{2-z} (e.g. C_{4-z}) heterocycloalkenyl (where z is the upper limit of the range) or a C_{7-z} heterocycloalkynyl group.

For the avoidance of doubt, the skilled person will understand that heterocyclyl groups that may form part of compounds of the invention are those that are chemically obtainable, as known to those skilled in the art. Various heterocyclyl groups will be well-known to those skilled in the art, such as 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, 2,3-dihydroisothiazolyl, dihydropyranyl, dihydropyridinyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), hexahydro-1H-thieno[3,4-d]imidazole, imidazolidinyl, imidazolinyl, isothiazolidinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuryl, tetrahydropyridinyl (such as 1,2,3,4-tetrahydropyridinyl and 1,2,3,6-tetrahydropyridinyl), thietanyl, thiiranyl, thiolanyl, tetrahydrothiopyranyl, thiomorpholinyl, trithianyl (including 1,3,5-trithianyl), tropanyl and the like.

Substituents on heterocyclyl groups may, where appropriate, be located on any atom in the ring system including a heteroatom. Further, in the case where the substituent is another cyclic compound, then the cyclic compound may be attached through a single atom on the heterocyclyl group, forming a spirocyclic compound. The point of attachment of heterocyclyl groups may be via any suitable atom in the ring system, including (where appropriate) a further heteroatom (such as a nitrogen atom), or an atom on any fused carbocyclic ring that may be present as part of the ring system. Heterocyclyl groups may also be in the N- or S-oxidised forms, as known to those skilled in the art.

At each occurrence when mentioned herein, particular heterocyclyl groups that may be mentioned include 3- to 8-membered heterocyclyl groups (e.g. a 4- to 6-membered heterocyclyl group, such as a 5- or 6-membered heterocyclyl group). Certain heterocyclyl groups that may be mentioned include hexahydro-1H-thieno[3,4-d]imidazole (particularly the (3aR,6aS)-hexahydro-1H-thieno[3,4-d]imidazole isomer thereof).

For the avoidance of doubt, references to polycyclic (e.g. bicyclic or tricyclic) groups (for example when employed in the context of heterocyclyl or cycloalkyl groups (e.g. heterocyclyl)) will refer to ring systems wherein at least two scissions would be required to convert such rings into a non-cyclic (i.e. straight or branched) chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, to groups in which two non-adjacent atoms are linked by an alkyl (which, when linking two moieties, may be referred to as alkylene) group (optionally containing one or more heteroatoms), which later groups may be referred to as bridged, or to groups in which the second ring is attached to a single atom, which latter groups may be referred to as spiro compounds.

As may be used herein, the term aryl may refer to C₆₋₁₄ (e.g. C₆₋₁₀) aromatic groups. Such groups may be monocyclic or bicyclic and, when bicyclic, be either wholly or partly aromatic. C₆₋₁₀ aryl groups that may be mentioned include phenyl, naphthyl, 1,2,3,4-tetrahydronaphthyl, indanyl, and the like (e.g. phenyl, naphthyl, and the like). For the avoidance of doubt, the point of attachment of substituents on aryl groups may be via any suitable carbon atom of the ring system.

For the avoidance of doubt, the skilled person will understand that aryl groups that may form part of compounds of the invention are those that are chemically obtainable, as known to those skilled in the art. Particular aryl groups that may be mentioned include phenyl and naphthyl, such as phenyl.

As may be used herein, references to heteroaryl (with may also be referred to as heteroaromatic) groups may refer to 5- to 14- (e.g. 5- to 10-) membered heteroaromatic groups containing one or more heteroatoms (such as one or more heteroatoms selected from oxygen, nitrogen and/or sulfur). Such heteroaryl groups may comprise one, two, or three rings, of which at least one is aromatic. Substituents on heteroaryl/heteroaromatic groups may, where appropriate, be located on any suitable atom in the ring system, including a heteroatom (e.g. on a suitable N atom).

The point of attachment of heteroaryl/heteroaromatic groups may be via any atom in the ring system including (where appropriate) a heteroatom. Bicyclic heteroaryl/heteroaromatic groups may comprise a benzene ring fused to one or more further aromatic or non-aromatic heterocyclic rings, in which instances, the point of attachment of the polycyclic heteroaryl/heteroaromatic group may be via any ring including the benzene ring or the heteroaryl/heteroaromatic or heterocyclyl ring.

For the avoidance of doubt, the skilled person will understand that heteroaryl groups that may form part of compounds of the invention are those that are chemically obtainable, as known to those skilled in the art. Various heteroaryl groups will be well-known to those skilled in the art, such as pyridinyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl, imidazopyrimidinyl, imidazothiazolyl, thienothiophenyl, pyrimidinyl, furopyridinyl, indolyl, azaindolyl, pyrazinyl, pyrazolopyrimidinyl, indazolyl, pyrimidinyl, quinolinyl, isoquinolinyl, quinazolinyl, benzofuranyl, benzothiophenyl, benzoimidazolyl, benzoxazolyl, benzothiazolyl, benzotriazolyl and purinyl.

For the avoidance of doubt, the oxides of heteroaryl/heteroaromatic groups are also embraced within the scope of the invention (e.g. the N-oxide).

As stated above, heteroaryl includes polycyclic (e.g. bicyclic) groups in which one ring is aromatic (and the other may or may not be aromatic). Hence, other heteroaryl groups that may be mentioned include groups such as benzo[1,3]dioxolyl, benzo[1,4]dioxinyl, dihydrobenzo[d]isothiazole, 3,4-dihydrobenz[1,4]oxazinyl, dihydrobenzothiophenyl, indolinyl, 5H, 6H,7H-pyrrolo[1,2-b]pyrimidinyl, 1,2,3,4-tetrahydroquinolinyl, thiochromanyl and the like.

For the avoidance of doubt, where a ring is depicted having circle therein, its presence shall indicate that the relevant ring is aromatic. Alternatively, aromatic groups may be depicted as cyclic groups comprising therein a suitable number of double bonds to allow for aromaticity.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. compounds of the invention in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more X groups are present, those X groups may be the same or different. Similarly, where two or more X groups are present and each represent C₁₋₃ alkyl, the C₁₋₃ alkyl groups in question (and any substituents thereon) may be the same or different.

For the avoidance of doubt, where groups are referred to herein as being optionally substituted it is specifically contemplated that such optional substituents may be not present (i.e. references to such optional substituents may be removed), in which case the optionally substituted group may be referred to as being unsubstituted.

Where used herein, a dashed bond (i.e. “- - -”, or the like) may indicate the position of attachment of the relevant substituent to the core molecule (i.e. the compound of the compound of formula I to which the substituent is attached).

For the avoidance of doubt, the skilled person will appreciate that compounds of the invention that are the subject of this invention include those that are obtainable, i.e. those that may be prepared in a stable form. That is, compounds of the invention include those that are sufficiently robust to survive isolation, e.g. from a reaction mixture, to a useful degree of purity.

In certain embodiments, the compound of formula I is not a compound as shown in Example 1 (i.e. the cationic compound as shown, in the presence of any anion, such as that shown).

Thus, in a certain alternative embodiment (e.g. a certain embodiment of the first aspect of the invention), there is the proviso that the following compound is excluded:
2-(5,11-dimethyl-6H-25-pyrido[4,3-b]carbazol-2-yl)-N,N-diethylethanamine,
optionally including pharmaceutically acceptable salts thereof.

In certain alternative embodiments, the compound of formula I is not a compound as shown in Examples 2 to 6 (i.e. the cationic compounds as shown, in the presence of any anion, such as that shown).

Thus, in certain alternative embodiments, the compound of formula I is not a compound as shown in Examples 1 to 6 (i.e. the cationic compounds as shown, in the presence of any anion).

As described herein, R^{A} represents C₁₋₆ alkyl, which is optionally substituted by one or more group selected from F, oxo (i.e. =O) and R^{A1}.

In particular embodiments, R^{A} may represent C₁₋₆ alkyl (e.g. C₁₋₃ alkyl) optionally substituted by:
one or more F;
up to one oxo; and
up to one R^{A1}.

For example, R^{A} may represent C₁₋₆ alkyl (e.g. C₁₋₃ alkyl), which is optionally substituted by one or more F and/or up to one R^{A1} (e.g. R^{A} represents C₁₋₃ alkyl optionally by substituted up to one R^{A1}).

In certain embodiments, R^{A} may represent C₁₋₆ alkyl (e.g. C₁₋₃ alkyl) optionally substituted by:
up to one oxo; and
up to one R^{A1} (e.g. up to one R^{A1}).

In a particular embodiment, R^{A} represents:
C₁₋₃ alkyl optionally substituted by one or more F; or
C₁₋₃ alkylene-R^{A1} optionally substituted by one or more F.

In a more particular embodiment, R^{A} represents:
C₁₋₃ alkyl (e.g. Me); or
C₁₋₃ alkylene-R^{A1} (e.g. C₂ alkylene-R^{A1}).

In a more particular embodiment, R^{A} represents:
C₁ alkyl; or
C₁₋₂ alkylene-R^{A1}.

In certain embodiments, R^{A} represents C₁ alkyl (i.e. Me).

In certain embodiments, R^{A} represents C₆ alkyl (e.g. linear hexyl).

In certain embodiments, R^{A} represents C₁₋₃ alkylene-R^{A1} optionally substituted by one or more F.

In certain embodiments, R^{A} represents C₁₋₂ alkylene-R^{A1} (e.g. C₂ alkylene-R^{A1}) optionally substituted by one or more F.

In particular embodiments, R^{A} represents C₁₋₂ alkylene-R^{A1}.

In alternative embodiments, R^{A} represents C₁₋₃ alkylene-R^{A1}.

For example, in certain embodiments R^{A} represents:
C₁₋₆ alkyl (e.g. methyl or hexyl, such as linear hexyl);
(CH₂)C(O)—R^{A1}; or
(CH₂)₂—R^{A1}.

In particular embodiments, R^{A} represents C₂ alkylene-R^{A1} (e.g. —(CH₂)₂—R^{A1}).

In particular embodiments, R^{A1} represents —N(R^{A3})R^{A4} or phenyl.

In particular embodiments, R^{A1} represents phenyl.

In certain embodiments that may be mentioned R^{A} represents:
C₁₋₆ alkyl (e.g. methyl or hexyl, such as linear hexyl);
(CH₂)C(O)—NH₂; or
(CH₂)₂-Ph.

In particular embodiments, R^{A1} represents —N(R^{A3})R^{A4}.

In certain embodiments, there is the proviso that R^{A3} and R^{A4} are not both H.

For example, in particular embodiments that may be mentioned, the compound of formula I is a compound of formula Ia:

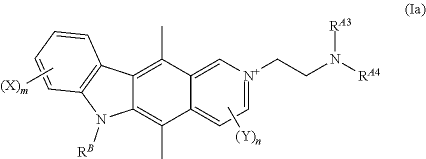

wherein R^{A3}, R^{A4}, R^{B}, m, n, X and Y are as described herein (i.e. for compounds of the first aspect of the invention, including all embodiments thereof).

In particular embodiments, R^{A3} and R^{A4} each independently represents H or C₁₋₃ alkyl (e.g. C₂ alkyl) optionally substituted with one or more F, optionally with the proviso that R^{A2} and R^{A3} are not both H, or alternatively R^{A3} and R^{A4} may be taken together to form a C₄₋₅ alkylene optionally substituted with one or more F.

In particular embodiments, R^{A3} and R^{A4} each independently represents H or C₁₋₃ alkyl (e.g. C₂ alkyl) optionally substituted with one or more F, with the proviso that R^{A2} and R^{A3} are not both H, or alternatively R^{A3} and R^{A4} may be taken together to form a C₄₋₅ alkylene optionally substituted with one or more F.

In particular embodiments, R^{A3} and R^{A4} each independently represents H or C₁₋₃ alkyl (e.g. C₂ alkyl), with the proviso that R^{A2} and R^{A3} are not both H, or alternatively R^{A3} and R^{A4} may be taken together to form a C₄₋₅ alkylene.

For the avoidance of doubt, where R^{A3} and R^{A4} are taken together to form a C₄₋₅ alkylene, such groups may be represented as follows:

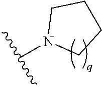

wherein q represents 1 or 2.

In particular embodiments (e.g. particularly for compounds of formula Ia), R^{A3} represents ethyl.

In particular embodiments (e.g. particularly for compounds of formula Ia), R^{A4} represents ethyl.

Thus, in particular embodiments (e.g. particularly for compounds of formula Ia), R^{A3} and R^{A4} each represent ethyl.

In particular embodiments, R^{A3} and R^{A4} are taken together to form a C₅ alkylene, which may be represented as follows:

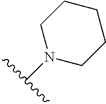 [SMILES: *N1CCCCC1]

Thus, in particular embodiments (e.g. particularly for compounds of formula Ia):
R^{A3} and R^{A4} each represent ethyl or
R^{A3} and R^{A4} are taken together to form a C₅ alkylene.

In particular embodiments (e.g. particularly for compounds of formula Ia), R^{B} represents H.

In particular embodiments (e.g. particularly for compounds of formula Ia), m and n each represent 0 or 1.

In more particular embodiments, m represents 0 or 1.

In particular embodiments, each X independently represents —OR^{A5}, —NH₂, halo, —CN or C₁₋₃ alkyl optionally substituted with one or more F.

In more particular embodiments, each X independently represents —OR^{A5}, —NH₂, halo (e.g. Cl or F, such as Cl), or C₁₋₃ alkyl (e.g. C₁₋₂ alkyl, such as Me) optionally substituted with one or more F.

In more particular embodiments, each X independently represents —OR^{A5}, —NH₂, halo (e.g. Cl or F, such as Cl), or C₁ alkyl optionally substituted with one or more F.

In more particular embodiments, each X independently represents —OR^{A5}, —NH₂, halo (e.g. Cl or F, such as Cl), or C₁ alkyl.

In more particular embodiments, each X independently represents —OR^{A5}, halo (e.g. Cl or F, such as Cl) or C₁ alkyl.

In more particular embodiments, each X independently represents —OR^{A5}, Cl or C₁ alkyl.

In particular embodiments, R^{A5} represents H or C₁ alkyl optionally substituted with one more F.

In particular embodiments, R^{A5} represents H or C₁ alkyl.

Thus, in particular embodiments, each X independently represents —OH, —OMe or Cl.

In particular embodiments, the compound of formula I is a compound of formula Ib:

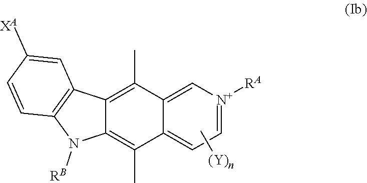

wherein R^{A}, R^{B}, m, n and Y are as described herein (i.e. for compounds of the first aspect of the invention, including all embodiments thereof) and X^{A} represents H or X as defined herein.

In particular embodiments, the compound of formula Ia is a compound of formula Ic:

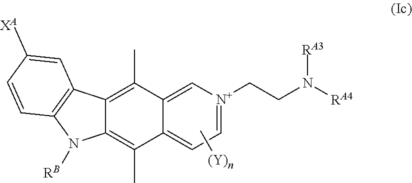

wherein R^{A3}, R^{A4}, R^{B}, m, n and Y are as described herein (i.e. for compounds of the first aspect of the invention, including all embodiments thereof) and X^{A} represents H or X as defined herein.

In more particular embodiments, m represents 0 (i.e. there is no X substituent present).

In more particular embodiments, n represents 0 (i.e. there is no Y substituent present).

Thus, in particular embodiments m and n are both 0.

For the avoidance of doubt, in a particular embodiment:
R^{A} represents C₁₋₃ alkylene-R^{A1} (e.g. C₂ alkylene-R^{A1});
R^{A1} represents —N(R^{A3})R^{A4};
R^{A3} represents ethyl;
R^{A4} represents ethyl;
R^{B} represents H;
m represents 0; and/or (e.g. and)
n represents 0.

For the avoidance of doubt, where compounds of the invention are present as a detectably labelled derivative thereof, such derivatives may also be in the form of a pharmaceutically-acceptable salt.

In particular embodiments, the compound of the invention is a compound of formula I or a pharmaceutically-acceptable salt thereof.

In alternative embodiments, the compound of the invention is a detectably labelled derivative of a compound of formula I, or a pharmaceutically-acceptable salt thereof. These may be useful in a number of applications, for example as imaging agents, in the immunoprecipitation of bound proteins, or in PROTAC development (see, for example, Hongying Gao, Xiuyun Sun, and Yu Rao, ACS Medicinal Chemistry Letters, 11(3), 237-240 (2020)).

The skilled person will be aware of numerous means for preparing detectably labelled derivatives of compounds as described herein. For example, where the compound of the invention is a detectably labelled derivative of a compound of formula I, or a pharmaceutically-acceptable salt thereof, the compound of formula I as defined may be further substituted by (i.e. a H group as present in such compounds may be replaced with) one or more (e.g. one) additional substituent forming a detectable label.

In particular embodiments, the one or more detectable label will be present as a substituent on a heteroatom, which heteroatom may be present in formula I (including all embodiments thereof) or as part of a substituent as defined for formula I, such as by replacing a H on a hydroxy or amine group.

In particular, such detectable labels may be present as alternative groups representing:
an R^{A} substituent (including an R^{A1} substituent (including R^{A2} and/or (e.g. or) R^{A3} substituent));
an R^{B} substituent;
one or more (e.g. one) substituent(s) representing an X group; and/or (e.g. or)
one or more (e.g. one) substituent(s) representing a Y group.

For the avoidance of doubt, in particular embodiments there is only one detectable label present in compounds of the invention (e.g. in particular embodiments, one of the above-mentioned groups may alternatively represent a detectable label).

Particular detectable labels that may be mentioned include those allowing for biotinylation of the compound of formula I (including all embodiments thereof), or pharmaceutically acceptable salt thereof, either directly (i.e. through direct substitution of the compound, such as by formation of an acid or amide with the carboxylic acid group of biotin), or via a linker group (which linker group may act as a substituent of the compound and be also bound to biotin via the carboxylic acid, such as through formation of an ester or amide thereof).

Further detectable groups (e.g. detectable labels) that may be mentioned include photoaffinity labels (PALs), such as azide groups.

Thus, in particular embodiments, the detectable label may be of formula -L-Z, wherein L represents either a direct bond or a suitable linker group and Z represents a detectable group.

The skilled person will understand that the term “detectable group”, as used herein, will refer to a chemical moiety the presence of which may be identified, and the quantity and, in certain instances, distribution thereof measured, using assays as known to those skilled in the art.

In particular embodiments, the detectable group is biotin, or an ester or amide derivative thereof (i.e. an ester or amide formed with the biotin carboxylic acid group).

For the avoidance of doubt, the structure of biotin is as indicated below:

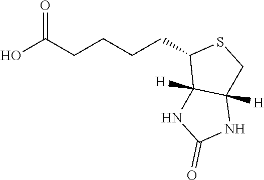 [SMILES: O=C(O)CCCC[C@@H]1SC[C@@H]2NC(=O)N[C@@H]21]

Thus, in particular embodiments Z (i.e. the detectable group) is of the following formula:

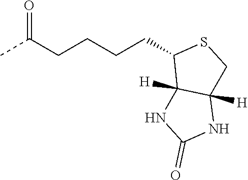

In particular embodiments, the detectable label is of the following formula:

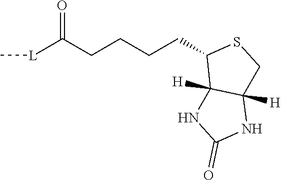

wherein L represents a direct bond or a suitable linker group.

In particular embodiments, L is a group of formula —(R^{a10}—W¹)ᵥ—, wherein:
each R^{a10} independently represents C₁₋₆ alkylene optionally substituted with one or more R^{b10};
W¹ represents —O— or —N(R^{c10})—;
each R^{b10} represents fluoro;
each R^{c10} independently represents H or C₁₋₃ alkyl optionally substituted with one or more fluoro;
v represents 1 to 6.

Thus, in particular embodiments the detectable label is of the following formula:

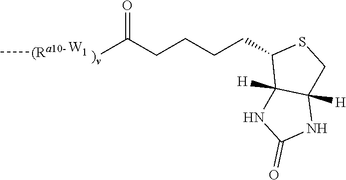

wherein R^{a10}, W¹ and v are as defined herein.

In particular embodiments, each R^{a10} represents C₁₋₃ alkylene, such as n-propylene.

In particular embodiments, each R^{c10} represents H.

In particular embodiments, at the point of attachment to Z, the relevant W¹ represents —NR^{c10}—, such as —NH—.

In alternative embodiments, L represents -L¹-L²- (wherein L¹ forms the point of attachment to the compound of formula I), wherein:
L¹ represents —O— or —NR^{d10}—;
L² represents a group as defined herein for L; and
R^{d10} represents C₁₋₃ alkyl (e.g. methyl) optionally substituted with one or more fluoro.

In alternative embodiments, Z may be represented by -L³-L⁴-Z¹, wherein:
L³ represents —C(O)—;
L⁴ represents C₁₋₆ alkylene (e.g. C₄ alkyl, such a n-butylene) optionally substituted with one or more fluoro; and
Z represents heterocyclyl (particularly wherein the heterocyclyl acts as a detectable group).

In alternative embodiments of the first aspect of the invention, the detectable label (or detectable group) may be a radioisotope. In such instances, the skilled person will understand that such radiolabelled compounds may be provided by replacement of one or more (e.g. one) of the atoms forming such compounds with a radioactive isotope thereof (such as by replacement of one or more H with the isotope tritium (T)), which may refer to enrichment of a sample of the compound so that a significant amount (e.g. at least 1%, at least 2%, at least 5% or at least 10% by weight, such as at least 20%, at least 30%, at least 40% or at least 50%, e.g. at least 90%) thereof has the relevant atom replaced with the radioisotope thereof.

Particular compounds of the invention that may be mentioned include those compounds as described in the examples provided herein (i.e. the cationic compounds as shown, in the presence of any anion, such as the anion as shown), and optionally pharmaceutically acceptable salts thereof.

### Medical Uses

As indicated herein, the compounds of the invention, and therefore compositions and kits comprising the same, are useful as pharmaceuticals.

Thus, according to a second aspect of the invention there is provided a compound of the invention, as hereinbefore defined (i.e. a compound as defined in the first aspect of the invention, including all embodiments and particular features thereof), for use as a pharmaceutical (or for use in medicine).

For the avoidance of doubt, references to compounds of the invention (i.e. compounds as defined in the first aspect of the invention) will include references to compounds of formula I (including all embodiments thereof) and pharmaceutically acceptable salts and detectably-labelled derivatives thereof.

In particular embodiments of the second aspect of the invention (including all alternatives thereof), there is a proviso that certain compounds are excluded, or that the compound of formula I is not selected from a list of certain compounds, as described in the first aspect of the invention.

In an alternative embodiment of the second aspect of the invention (including all alternatives thereof), there is no proviso that certain compounds are excluded. In other words, it is contemplated that the compound of formula I, 2-(5,11-Dimethyl-6H-25-pyrido[4,3-b]carbazol-2-yl)-N,N-diethylethanamine, and pharmaceutically acceptable salts and detectably-labelled derivatives thereof, is provided for use as a pharmaceutical (or for use in medicine).

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the active compounds of the invention to which they are metabolised), may also be described as “prodrugs”.

For the avoidance of doubt, compounds of the invention are therefore useful because they possess pharmacological activity, and/or are metabolised in the body following oral or parenteral administration to form compounds that possess pharmacological activity.

Compounds may also be useful in medicine as diagnostic agents (particularly compounds comprising datable labels, as described herein).

As described herein, compounds of the invention may be particularly useful in treating cancers, particularly cancers characterised by increased activity of the MYC pathway.

Thus, in a third aspect of the invention, there is provided a compound of the invention, as hereinbefore defined, for use in the treatment of a cancer, such as a characterised by increased activity of the MYC pathway.

In an alternative third aspect of the invention, there is provided a method of treating a cancer, such as a cancer characterised by increased activity of the MYC pathway, comprising administering to a patient in need thereof a therapeutically effective amount of a compound of the invention, as hereinbefore defined.

In a further alternative third aspect of the invention, there is provided the use of a compound of the invention, as hereinbefore defined, for the manufacture of a medicament for the treatment of a cancer, such as a characterised by increased activity of the MYC pathway.

The skilled person will understand that references to increased activity of the MYC pathway may refer to any instances where cancers have, in a significant number of cells thereof (e.g. at least 10%, such as at least 30%, at least 50%, at least 70%, or at least 90%) biological activity indicative of increased MYC activity when compared to the MYC activity observed in corresponding non-cancerous cells.

In such instances, the term “increased” may refer to the presence of MYC activity where such activity would not normally occur in corresponding non-cancerous cells, or to an increase in the level of activity when compared to corresponding non-cancerous cells, such as an at least 10% increase (e.g. an at least 50% increase or, more typically, an at least 100% increase). Such increased MYC activity may be observed in samples of such cancerous cells, and thus measured in relation to the given sample as a whole.

The skilled person will be aware that increased MYC activity may for instance refer to an increase in the expression of the MYC gene. For the avoidance of doubt, references to the MYC gene may include references to the MYC, MYCN and MYCL oncogenes.

The skilled person will also be aware that such increased MYC activity may result from a range of oncogenic alterations, such as:
increased copy number of the MYC, MYCN or MYCL oncogenes (which may be referred to as “copy number gain” or “gene amplification”);
elevated/deregulated expression of MYC, MYCN or MYCL mRNA/protein; and/or increased activity of MYC-family proteins.

For the avoidance of doubt, references to increased copy number (i.e. gene amplification) of MYC-family (MYC, MYCN, MYCL) genes refer to the presence of additional copies of the relevant MYC gene in the genome of the cancerous cells when compared the corresponding non-cancerous cell-type.

The skilled person will be aware that increased activity of the MYC pathway, including increased copy number, can be identified and, if necessary, measured using techniques routine to those skilled in the art (such as those described in The Myc Gene: Methods and Protocols, Soucek and Sodir (Ed.), Methods in Molecular Biology 1012; Springer Protocols, Humana Press, Library of Congress Control Number: 2013945869 (2013)).

The skilled person will be aware that copy number can be measured in situ by using techniques such as FISH (fluorescence in situ hybridization), NGS (next generation sequencing), or array-based analysis of copy number variations. The results obtained can be compared to copy number in corresponding normal (i.e. non-cancerous) cells. (e.g. where the cancer is of blood cells, the copy number in non-cancerous blood cells, such as those obtained from the same patient).

References to increased MYC activity may also refer to elevated MYC-family mRNA levels (overexpression) or to “deregulated” expression (meaning that the gene is expressed in the wrong cell at the wrong time). Expression changes can be caused by gene amplification (increased gene copy number) or, for instance, by altered cell signaling. Determination of whether MYC is overexpressed and/or deregulated may be made by comparison to a control sample. This control sample could be of normal (non-cancerous) cells from the same tissue, of benign tumors from the same tissue and/or of malignant tumours of lower grade. Levels of MYC mRNA expression be measured by RNA sequencing, RT-QPCR, microarray analysis, or by in situ hybridization.

As described herein, references to increased MYC activity may also refer to elevated and/or deregulated MYC-family protein levels. The skilled person will understand that such protein levels can be measured by, for instance, immunohistochemistry, western blot, RPPA (Reverse Phase Protein Array) and/or mass spectrometry. The skilled person will also understand that some tumours with low MYC mRNA might still have high protein levels due to for instance protein stabilization or increased MYC mRNA translation.

The skilled person will also understand that increased MYC activity may arise due to posttranslational modifications and/or altered protein interactions in response to cell signalling, resulting in the activity rather than the level of the MYC proteins being increased. This results in activation (or repression) of MYC target genes, and can be measured using routine techniques, such as expression profiling of mRNA and/or protein by RT-QPCR, microarray analysis, RNA sequencing, western blot, protein arrays, and/or mass spectrometry.

For the avoidance of doubt, the above-mentioned factors resulting in increased MYC activity also be collectively referred to as “activation of the MYC pathway” or the like.

The skilled person will understand that references to the treatment of a particular condition (or, similarly, to treating that condition) will take their normal meanings in the field of medicine. In particular, the terms may refer to achieving a reduction in the severity and/or frequency of occurrence of one or more clinical symptom associated with the condition, as adjudged by a physician attending a patient having or being susceptible to such symptoms. For example, in the case of the treatment of a cancer, the term may refer to achieving a reduction (e.g. at least a 5% reduction, such as at least a 10% or 20% reduction) in the number of cancer cells present and/or the volume of tumor mass (in the case of a solid tumor).

As used herein, references to a patient (or to patients) will refer to a living subject being treated, including mammalian (e.g. human) patients. In particular, references to a patient will refer to human patients.

For the avoidance of doubt, the skilled person will understand that such treatment will be performed in a patient (or subject) in need thereof. The need of a patient (or subject) for such treatment may be assessed by those skilled the art using routine techniques.

As used herein, the terms disease and disorder (and, similarly, the terms condition, illness, medical problem, and the like) may be used interchangeably.

As used herein, the term effective amount will refer to an amount of a compound that confers a therapeutic effect on the treated patient. The effect may be observed in a manner that is objective (i.e. measurable by some test or marker) or subjective (i.e. the subject gives an indication of and/or feels an effect). In particular, the effect may be observed (e.g. measured) in a manner that is objective, using appropriate tests as known to those skilled in the art.

Compounds of the invention may find particular utility in the treatment of cancers known to be frequently characterised by activation of the MYC pathway. Thus, in certain embodiments, the cancer is a cancer (or a combination of one or more cancers) selected from the list consisting of:
Burkitt's lymphoma;
ovarian cancer, such as ovarian cancer with BRCA alterations;
basal-like/triple negative breast cancer;
esophageal squamous cell carcinoma;
colon cancer;
endometrial cancer;
neuroblastoma;
small cell lung carcinoma;
medulloblastoma, in particular group 3;
pancreatic cancer;
head and neck cancer;
prostate cancer; and
hepatocellular carcinomas.

### Pharmaceutical Compositions

As described herein, compounds of the invention are useful as pharmaceuticals. Such compounds may be administered alone or may be administered by way of known pharmaceutical compositions/formulations.

In a fourth aspect of the invention, there is provided a pharmaceutical composition comprising a compound of the invention as defined herein, and optionally one or more pharmaceutically-acceptable excipient.

In particular embodiments of the fourth aspect of the invention, there is a proviso that certain compounds are excluded, or that the compound of formula I is not selected from a list of certain compounds, as described in the first or second (e.g. the second) aspect of the invention.

However, in a more particular embodiment of the fourth aspect of the invention (including all alternatives thereof), there is no proviso that certain compounds are excluded. In other words, it is contemplated that the compound of formula I, 2-(5,11-Dimethyl-6H-25-pyrido[4,3-b]carbazol-2-yl)-N,N-diethylethanamine, and pharmaceutically acceptable salts and detectably-labelled derivatives thereof, is provided as part of a pharmaceutical composition.

As used herein, the term pharmaceutically-acceptable excipients includes references to vehicles, adjuvants, carriers, diluents, pH adjusting and buffering agents, tonicity adjusting agents, stabilizers, wetting agents and the like. In particular, such excipients may include adjuvants, diluents or carriers.

For the avoidance of doubt, references herein to compounds of invention being for particular uses (and, similarly, to uses and methods of use relating to compounds of the invention) may also apply to pharmaceutical compositions comprising compounds of the invention, as described herein.

Thus, in a fifth aspect of the invention, there is provided a pharmaceutical composition as defined in the fourth aspect of the invention (including all embodiments thereof) for use in the treatment of a cancer characterised by activation of the MYC pathway (as defined herein, with reference to the third aspect of the invention and all embodiments thereof).

The skilled person will understand that compounds of the invention may act systemically and/or locally (i.e. at a particular site), and may therefore be administered accordingly using suitable techniques known to those skilled in the art.

The skilled person will understand that compounds and compositions as described herein will normally be administered orally, intravenously, subcutaneously, intratumorally, buccally, rectally, dermally, nasally, tracheally, bronchially, sublingually, intranasally, topically, by any other parenteral route or via inhalation, in a pharmaceutically acceptable dosage form.

Pharmaceutical compositions as described herein will include compositions in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration.

Thus, in particular embodiments, the pharmaceutical formulation is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

Depending on e.g. potency and physical characteristics of the compound of the invention (i.e. active ingredient), pharmaceutical formulations that may be mentioned include those in which the active ingredient is present in an amount that is at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The skilled person will understand that compounds of the invention may be administered (for example, as formulations as described hereinabove) at varying doses, with suitable doses being readily determined by one of skill in the art. Oral, pulmonary and topical dosages (and subcutaneous dosages, although these dosages may be relatively lower) may range from between about 0.01 μg/kg of body weight per day (μg/kg/day) to about 200 μg/kg/day, preferably about 0.01 to about 10 μg/kg/day, and more preferably about 0.1 to about 5.0 μg/kg/day. For example, when administered orally, treatment with such compounds may comprise administration of a formulations typically containing between about 0.01 μg to about 2000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. In some instances, treatment may comprise administration of such compounds and compositions in a single daily dose, or the total daily dosage may be administered in divided doses of two, three or four times daily (e.g. twice daily with reference to the doses described herein, such as a dose of 25 mg, 50 mg, 100 mg or 200 mg twice daily).

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

For the avoidance of doubt, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. Although the above-mentioned dosages are exemplary of the average case, there can, of course, be individual instances where higher or lower dosage ranges are merited, and such doses are within the scope of the invention.

### Combinations and Kits-of-Parts

The skilled person will understand that treatment with compounds of the invention may further comprise (i.e. be combined with) further treatment(s) for the same condition. In particular, treatment with compounds of the invention may be combined with means for the treatment of cancers (such as a type of cancer as described herein, e.g. cancers characterised by activation of the MYC pathway), such as treatment with one or more other therapeutic agent that is useful in the in the treatment of such cancers and/or one or more physical method used in the treatment of such cancers (such as treatment through surgery), as known to those skilled in the art.

As described herein, compounds of the invention may also be combined with one or more other (i.e. different) therapeutic agents (i.e. agents that are not compounds of the invention) that are useful in the treatment of cancers, such as those cancers described herein. Such combination products that provide for the administration of a compound of the invention in conjunction with one or more other therapeutic agent may be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the one or more other therapeutic agent).

Thus, according to a sixth aspect of the invention, there is provided a combination product comprising:
(I) a compound of the invention, as hereinbefore defined (i.e. in the first aspect of the invention, including all embodiments and particular features thereof); and
(II) one or more other therapeutic agent that is useful in the treatment of cancer (such as a cancer as described herein),
wherein each of components (I) and (II) is formulated in admixture, optionally with one or more a pharmaceutically-acceptable excipient.

In a seventh aspect of the invention, there is provided a kit-of-parts comprising:
(a) a pharmaceutical formulation as hereinbefore defined (i.e. in the fifth aspect of the invention); and
(b) one or more other therapeutic agent that is useful in the treatment of cancer (such as a cancer as described herein), optionally in admixture with one or more pharmaceutically-acceptable excipient,
which components (a) and (b) are each provided in a form that is suitable for administration in conjunction (i.e. concomitantly or sequentially) with the other.

In particular embodiments of the sixth and seventh aspects of the invention, there is a proviso that certain compounds are excluded, or that the compound of formula I is not selected from a list of certain compounds, as described in the first or second (e.g. the second) aspect of the invention.

However, in a more particular embodiment of the sixth and seventh aspects of the invention (including all alternatives thereof), there is no proviso that certain compounds are excluded. In other words, it is contemplated that the compound of formula I, 2-(5,11-Dimethyl-6H-25-pyrido[4,3-b]carbazol-2-yl)-N,N-diethylethanamine, and pharmaceutically acceptable salts and detectably-labelled derivatives thereof, is provided as part of a combination product (according to the sixth aspect of the invention) or as part of a kit-of-parts (according to a seventh aspect of the invention).

With respect to the kits-of-parts as described herein, by “administration in conjunction with” (and similarly “administered in conjunction with”) we include that respective formulations are administered, sequentially, separately or simultaneously, as part of a medical intervention directed towards treatment of the relevant condition.

Thus, in relation to the present invention, the term “administration in conjunction with” (and similarly “administered in conjunction with”) includes that the two active ingredients (i.e. a compound of the invention and a further agent for the treatment of cancer, or compositions comprising the same) are administered (optionally repeatedly) either together, or sufficiently closely in time, to enable a beneficial effect for the patient, that is greater, over the course of the treatment of the relevant condition, than if either agent is administered (optionally repeatedly) alone, in the absence of the other component, over the same course of treatment. Determination of whether a combination provides a greater beneficial effect in respect of, and over the course of, treatment of a particular condition will depend upon the condition to be treated, but may be achieved routinely by the skilled person.

Further, in the context of the present invention, the term “in conjunction with” includes that one or other of the two formulations may be administered (optionally repeatedly) prior to, after, and/or at the same time as, administration of the other component. When used in this context, the terms “administered simultaneously” and “administered at the same time as” includes instances where the individual doses of the compound of the invention and the additional compound for the treatment of cancer, or pharmaceutically acceptable salts thereof, are administered within 48 hours (e.g. within 24 hours, 12 hours, 6 hours, 3 hours, 2 hours, 1 hour, 45 minutes, 30 minutes, 20 minutes or 10 minutes) of each other.

Other therapeutic agents useful in the treatment of cancers (such as those cancers as described herein) will be well-known to those skilled in the art. For example, such other therapeutic agents may include therapeutic agents routinely used in the treatment of the relevant cancer type, as will be known to those skilled in the art.

### Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable excipient.

In further aspects of the invention, there is provided a process for the preparation of a combination product or kit-of-parts as hereinbefore defined, which process comprises bringing into association a compound of the invention, as hereinbefore defined, with the other therapeutic agent that is useful in the treatment of the relevant disease or disorder, and at least one pharmaceutically-acceptable excipient.

As used herein, references to bringing into association will mean that the two components are rendered suitable for administration in conjunction with each other.

Thus, in relation to the process for the preparation of a kit-of-parts as hereinbefore defined, by bringing the two components “into association with” each other, we include that the two components of the kit-of-parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention as described herein may be prepared in accordance with techniques that are well known to those skilled in the art.

In particular, compounds of formula I may be obtained by analogy with the literature processes for the synthesis of 2-(5,11-dimethyl-6H-25-pyrido[4,3-b]carbazol-2-yl)-N,N-diethylethanamine acetate, using conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

Furthermore, compounds of formula I may be obtained by analogy with the procedure shown in Compound Example 1 for the synthesis of 2-(5,11-dimethyl-6H-25-pyrido[4,3-b]carbazol-2-yl)-N,N-diethylethanamine bromide. In addition, compounds of formula I may be obtained by salt exchange of that compound using conventional synthetic procedures, in accordance with standard techniques.

The skilled person will understand that the substituents as defined herein, and substituents thereon, may be modified one or more times, after or during the processes described above for the preparation of compounds of the invention by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999), the contents of which are incorporated herein by reference.

Without wishing to be bound by theory, it is believed that compounds of the invention are able to treat cancers, particularly those cancers characterised by increased MYC activity, such as may be due to gene copy number alterations and/or increased expression/activity of MYC (i.e. activation of the MYC pathway), based on their ability to act as potent and specific inhibitors of MYC:MAX interaction, thus inhibiting MYC-dependent tumor cell growth.

It is also believed that detectably-labelled derivatives of compounds of the invention may be useful as diagnostic agents and/or as research tools, such as in drug development.

Compounds of the invention may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

### BRIEF DESCRIPTION OF THE FIGURES

FIG. 1: MYCMI-7 (Compound Example 1) inhibits the MYC:MAX interaction in cells, bind MYC in vitro, and blocks MYC function. A) Molecular structure of MYCMI-7. B-E) split Gaussia luciferase (GLuc) assay. MYC or MYCN and MAX Gluc constructs were co-transfected into HeLa cells together with a firefly luciferase vector. 24 hours post transfection cells were treated with compounds, and after another 17-24 hours cells were harvested and both GLuc and firefly luciferase activities were measured. The mean of three different experiments is shown. Treatment with: B) 5 μM of MYCMI-7, 64 μM 10058-F4 or 5 μM JQ1, respectively, C) 6.5 μM MYCMI-7 on cells expressing MYC:MAX GLuc vs. GCN4:GCN4 GLuc, D) 5 μM MYCMI-7 and 5 μM ellipticine. E) 5 μM MYCMI-7 for various time points. F) in situ proximity ligation assay (isPLA). MCF7 cells were treated with 5 μM MYCMI-7 for 24 hours, thereafter fixed and subjected to the isPLA assay to detect endogenous MYC:MAX and FRA1:JUN interactions, respectively. MYC:GAL4 was used as negative control in the isPLA assay. G) Quantification of isPLA dots. (H) Co-immunoprecipitation (co-IP) of endogenous MYC and MAX proteins from MCF7 cells after treatment with 5 μM MYCMI-7 at various times. IP=immunoprecipitation; W=western blot. I) Chromatin immunoprecipitation (ChIP) of MYC at the ODC1 target gene promoter after treatment of cells with 5 μM MYCMI-7 for 6 hours. J) MYC target gene ODC1 expression after treatment with 5 μM MYCMI-7, quantified by qPCR. K) ChIP of MYC at the ODC promoter in MCF7 cells after 5 μM MYCMI-7 treatment for indicated time points. L) Recombinant MYC bHLHZip protein domain immobilized onto a CM5 chip was subjected to various concentrations of MYCMI-7 and the complex of MYCbHLHZip-MYCMI-7 was measured as response units by SPR. The sensogram was produced by subtracting the blank response.

FIG. 2: MYCMI-7 downregulates MYC and MYCN protein expresión. A) Steady state levels of MYC protein in MCF7 cells after 17 hours exposure to indicated concentrations MYCMI-7 or ellipticine B) MYC mRNA expression in MCF7 cells after treatment with MYCMI-7 for various time points. C) Steady state levels of MYC protein in different HCT116 cell lines (WT, FBW7−/− or p53−/−) after 17 hours exposure to 5 μM MYCMI-7. Endogenous MYC was immunoprecipitated and detected by western blot analysis. D) Cycloheximide (CHX) chase in HCT116 cells. Cells were subjected to 5 μM MYCMI-7 30 min prior cycloheximide addition. Cells were harvested after the indicated time points, MYC was immunoprecipitated using N262 antibody and the MYC levels analyzed using western blot (C33 antibody). E) CHX chase after MYCMI-7 treatment performed as in (D) using Fbxw7-deficient HCT116 cells. F) Stable expression of wt, T58A or S62A MYC in HO15.19 MYC−/− cells. The cells were treated with 5 μM MYCMI-7 overnight, and MYC expression was analyzed by western blot. G) Cartoon showing a linear structural representation of wt MYC and indicated mutants. H-J) Expression of MYC mutants after MYCMI-7 treatment. COS-7 cells were transfected with CMV-driven vectors containing HA-tagged wt MYC or mutants for 24 hrs and treated with 5 μM MYCMI-7 for 17 hrs. The DN (H) and DC (I) MYC mutants encodes the C-terminal and N-terminal halves of the MYC protein, respectively. DBr (J) lacks the DNA-binding basic region of the protein. A-J). Representative western blots out of at least three experiments are shown.

FIG. 3: MYCMI-7 reduces tumor cell growth/viability in a MYC-dependent manner and down-regulates the MYC pathway. A) Immortalized Rat1 fibroblasts with different MYC status; TGR1 (wt MYC), HO15.19 (MYC−/−) and HOMyc3 (HO15.19 with reconstituted MYC) were treated with indicated concentrations of MYCMI-7 for 48 hours. Cell proliferation/viability/metabolic activity was measured using the WST-1 assay. B) Human neuroblastoma cells with or without MYCN-amplification where treated with 6.25 μM MYCMI-7 or 64 μM 10058-F4 for 24 hrs. C) 3D cultures of SKN-DZ neuroblastoma cells with MYCN-amplification exposed to indicated concentrations of MYCMI-7 for 2 weeks. Colony formation in agarose was detected by MTT staining and quantified. D) Correlation of MYCMI-7 response (GI₅₀) with MYC mRNA levels of the NCI-60 human tumor cell lines extracted from CellMiner™ and complemented with MYC protein levels from the Novartis proteome scout project or from the literature (Castell A, et al. (2018) A selective high affinity MYC-binding compound inhibits MYC:MAX interaction and MYC-dependent tumor cell proliferation. Sci Rep 8(1):10064). “Responsive” and “less responsive”; cell lines with positive and negative log 10 GI₅₀ values, respectively. “Higher MYC” and “lower MYC”; cell lines with higher and lower MYC expression levels (MYC mRNA/protein) than average, respectively. For the WST-1 assays the average of three different experiments performed in triplicates is shown. E) Gene set enrichment analysis (GSEA) of RNA-seq data from MCF7 breast carcinoma cells treated with 5 μM MYCMI-7 for 24 hrs. F) Inhibition of MYC transactivation of target genes CR2, RSG16, CAKMV and nucleolin as determined by RT-qPCR analysis, based on three biological experiments with three technical repeats each. U2OS-MYCER cells were treated with (MYC ON) or without (MYC OFF) 100 nM 4-hydroxy-tamoxifen (4-OHT) for 4 hours, after which DMSO or MYCMI-7 (5 μM) were added for 24 hours before total RNA was extraction. Fold changes in mRNA expression are presented relative to DMSO in MYC OFF cells after normalization to GAPDH, used as reference gene.

FIG. 4: MYCMI-7 induces apoptosis in malignant cells and growth arrest in normal cells. A) Cell cycle distribution analysis by flow cytometry of MYC-regulated P493-6 cells and normal rat embryo fibroblasts (REFs). Left panel, P493-6 synchronized in G0 by 24 hours treatment of 1 mg/ml doxycycline. Doxycycline was removed and the cells were harvested 48 hours after the addition of fresh medium containing DMSO or 5 μM MYCMI-7. Right panel, flow cytometry analysis of normal REFs treated with DMSO or 5 μM MYCMI-7. B) Apoptosis assay using the Cell Death Detection ELISA^{plus} kit. Left panel, immortalized Rat1 fibroblasts with different MYC status after 24 hours of 5 μM MYCMI-7 treatment, middle panel and right panels, normal REFs and normal peripheral blood lymphocytes, respectively, after 24 and 48 hours of 5 μM MYCMI-7 treatment. C) Measurement of viability and apoptosis by Apo-Tox Glo Triplex assay in A375 melanoma cells after 3 days of treatment with MYCMI-7 with the indicated concentrations. D) Normal human dermal fibroblasts (NHDFs) exposed to indicated concentrations of MYCMI-7 for 72 hrs after which metabolic activity was measured by resazurin assay and cell number measured by in a cell counter. E) NHDFs treated with 3 μM MYCMI-7 for 72 hrs. Viability was measured by trypan blue exclusion assay. F) Cell growth measured with Cyt62 assay of primary normal human epidermal melanocytes (NHEM) treated for 72 hours with MYCMI-7, G) Viability measured by trypan blue exclusion assay of NHEM after treatment for 72 hours with MYCMI-7. A-G) One representative biological experiment out of at three, performed in triplicates is shown.

FIG. 5: MYCMI-7 does not induce DNA damage signaling at active concentrations and acts independently of topoisomerase 2 and p53. A) Measurement of MYC mRNA expression by RTqPCR after treatment with MYCMI-7 and different topoisomerase inhibitors at indicated concentrations. B-C) Auxin-induced depletion of topoisomerase 2A (TOP2A) in HCT116 cells does not affect B) MYC:MAX interactions measured by isPLA, or C) sensitivity to MYCMI-7 as determined by resazurin assay after titration of MYCMI-7 concentrations is the presence and absence of TOP2A. D) Western blot analysis of p53 and p21 expression after exposure to 5 μM MYCMI-7, ellipticine or camptothecin (CPT) in HCT116 cells. E) Western blot analysis of phospho-ATM, total ATM and p53 expression after exposure to indicated concentrations of MYCMI-7 and ellipticine in A375 cells after 3 hrs treatment. F) p53 knockout does not affect sensitivity to MYCMI-7 in HCT116 cells as determined by WST1 assay after titration of MYCMI-7 concentrations.

FIG. 6: MYCMI-7 is a potent inhibitor of growth of primary patient derived glioblastoma and AML tumor cells ex vivo. A) Primary glioblastoma samples from 42 patient biopsies were cultured for 72 hours in presence of MYCMI-7 at indicated doses and cell growth was measured by the resazurin assay. B) IC₅₀ values for myeloid cell lines and primary AML cells from patient biopsies treated with MYCMI-7 at different concentrations in cell culture and assayed by the WST-1 assay. C) Primary AML cells from patient biopsies where cultured for 72 hours in presence of MYCMI-7 at indicated doses cell growth was monitored by the WST-1 assay. One representative biological experiment out of at three, performed in triplicates is shown.

FIG. 7: MYCMI-7 inhibits MYC/BCL-XL-driven acute myeloblastic leukemia in an allogenic mouse model. A) Schematic plan of the experiment B) Flow cytometry analysis of GFP⁺ leukemic blasts in bone marrow on day 11 (n=5), day 15 (n=5), and at the end point (n=9) after daily i.p. injection of 12.5 mg/kg MYCMI-7, compared with vehicle (n=3) mice. C). H&E and MYC immunohistochemistry (IHC) stainings of spleen sections at the endpoint. Images were taken at 10× magnification. D) quantification of MYC IHC stainings of spleen sections at the endpoint. E) Western blot analysis of cleaved caspase 3 and H3K9me3 expression in spleens at endpoint after treating mice with MYCMI-7 or vehicle (3 mice from each condition). Actin was used as loading control. At the end points, the last injection was done 3 hrs before sacrifice.

FIG. 8: MYCMI-7 inhibits tumor growth and prolongs survival in breast cancer and neuroblastoma xenograft experiments. A) Tumor growth of MDA-MB-231 xenografts in mice injected with 6.25 mg/kg MYCMI-7 or vehicle intratumorally twice a week until sacrificed. Tumor growth was monitored by measuring tumor size (mm³) every 4th day. B) Survival curve of the mice in A) sacrificed when the tumors reached a volume of 1000 mm³. C) Immunohistochemistry staining for MYC and apoptosis marker caspase 3 in MDA-MB-231 tumor tissue from vehicle- and MYCMI-7 treated animals at end point, D) Immunofluorescence staining of proliferation and apoptosis markers Ki67 and TUNEL, respectively, in MDA-MB-231 xenograft tumor tissue from vehicle- and MYCMI-7 treated animals at end point. E) MYCN-amplified SK-N-DZ neuroblastoma xenograft mice injected with 6.25 mg/kg MYCMI-7 or vehicle intratumorally twice a week until sacrificed. Tumor growth was monitored by measuring tumor volume (ml) daily. F) Survival curve of mice in E) sacrificed at a tumor volume of 1500 mm³. G-H) H&E (G) and MYCN IHC (H) stainings of SK-N-DZ xenograft tumor tissue from vehicle- and MYCMI-7 treated animals at end point. A-H) At the end points, the last injection was done 3 hrs before sacrifice. Images were taken at 10× or 40× magnification.

FIG. 9: Experiments reported in FIG. 3A and FIG. 1L were repeated using the same techniques for the compounds of Examples 2 to 6 in MYC wt (TGR), MYC knockout (HO1519) and MYC-reconstituted (Myc3) rat cells (left) and by SPR (right). Examples 2 to 6 reduce tumor cell growth/viability in a MYC-dependent manner and bind recombinant MYC.

FIGS. 10A and B: Experiments reported in FIG. 3A and FIG. 1L were repeated using the same techniques for the compounds of Examples 7 to 9 in MYC wt (TGR) and MYC knockout (HO1519) rat cells (left) and by SPR (right). Examples 7 to 10 reduce tumor cell growth/viability in a MYC-dependent manner and bind recombinant MYC.

### EXAMPLES

The present invention will be further described by reference to the following examples, which are not intended to limit the scope of the invention.

2-(5,11-Dimethyl-6H-25-pyrido[4,3-b]carbazol-2-yl)-N,N-diethylethanamine acetate (which is also referred to herein as MYCMI-7) was synthesized by Honghui-Meditech, China, purity >95% by HPLC. DMSO, JQ1, 10058-F4, ellipticine, doxorubicin, etoposide and camptothecin were purchased from Sigma-Aldrich. All compounds were dissolved in DMSO (Sigma-Aldrich) to a final concentration of 10 μM, verified by mass spectrometry (LC-MS) and stored in −80° C. for further use.

### Abbreviations

Abbreviations as used herein will be known to those skilled in the art. In particular, the following abbreviations may be used herein.
eq equivalents
Et₃N triethylamine
H₂O water
MeOH methanol
rt room temperature

### EXAMPLE COMPOUNDS

In the event that there is a discrepancy between nomenclature and any compounds depicted graphically, then it is the latter that presides (unless contradicted by any experimental details that may be given or unless it is clear from the context).

### Compound Example 1 (also referred to herein as MYCMI-7): 2-(5,11-Dimethyl-6H-25-pyrido[4,3-b]carbazol-2-yl)-N,N-diethylethanamine bromide

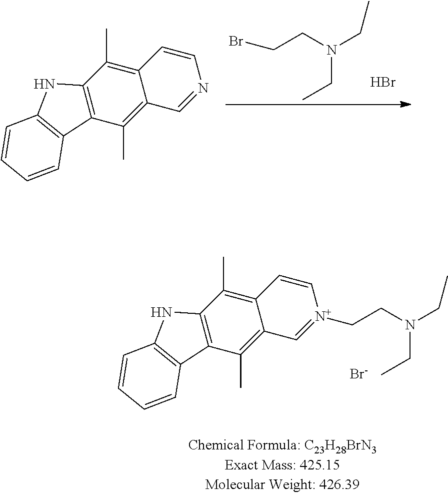

(N,N-diethylamino)ethyl bromide hydrobromide (1.08 eq) followed by Et₃N-MeOH solution (1.08 eq) was added to a solution of ellipticine (0.5 g) in MeOH (83 mL) at rt. After stirring at rt for 20 h, (N,N-diethylamino)ethyl bromide hydrobromide (0.25 eq) and Et₃N-MeOH solution (0.25 eq) were added to the reaction mixture. After stirring at rt for 5 h, (N,N-diethylamino)ethyl bromide hydrobromide (0.25 eq) and Et₃N-MeOH solution (0.25 eq) were added to the reaction mixture. The mixture was stirred overnight, the solvent removed in vacuo, the obtained solid dissolved in H₂O, filtered, and recrystallized from EtOH (95%). The solids were collected to give the title compound (10 g, >95%).

For the avoidance of doubt, Compound Example 1 (excluding the anion as indicated but in the presence of a suitable anion, such as bromide or acetate, e.g. bromide) may also be referred to herein as MYCMI-7.

Unless otherwise specified, Compound Example 1 (MYCMI-7) may be used in biological examples as described herein in the form of the bromide or acetate salt (i.e. having a Br⁻ or AcO⁻ counterion).

### Compound Examples 2-6

The following example compounds were obtained from compound library of the NCI (National Cancer Institute, Bethesda):

| Ex- | | NCI Library |
|---|---|---|
| ample | Structure | No. |
| 2 | 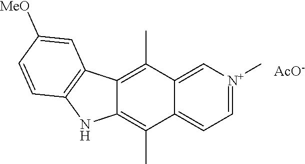 [SMILES: "AcO-" ; COc1ccc2[nH]c3c(C)c4cc[n+](C)cc4c(C)c3c2c1] | NSC627505 |
| 3 | 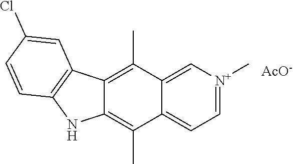 [SMILES: "AcO-" ; Cc1c2cc[n+](C)cc2c(C)c2c1[nH]c1ccc(Cl)cc12] | NSC632855 |
| 4 | 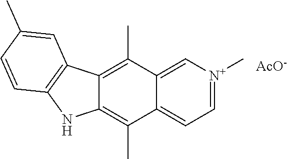 [SMILES: "AcO-" ; Cc1ccc2[nH]c3c(C)c4cc[n+](C)cc4c(C)c3c2c1] | NSC639364 |
| 5 | 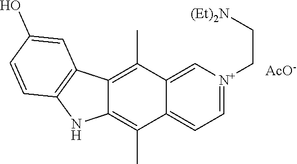 | NSC311152 |
| 6 | 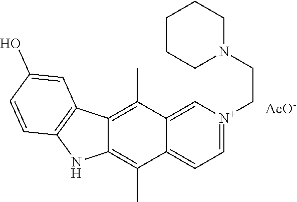 [SMILES: "AcO-" ; Cc1c2cc[n+](CCN3CCCCC3)cc2c(C)c2c1[nH]c1ccc(O)cc12] | NSC311153 |

Compound Examples 2 to 6 may be prepared using techniques analogous to those used in the preparation of Compound Example 1.

### Compound Example 7: 2,5,11-Trimethyl-6H-pyrido[4,3-b]carbazol-2-ium iodide

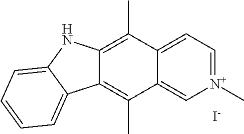 [SMILES: Cc1c2cc[n+](C)cc2c(C)c2c1[nH]c1ccccc12 ; [I-]]

To a suspension of 5,11-dimethyl-6H-pyrido[4,3-b]carbazole (11.0 mg, 0.045 mmol) in acetone (0.8 mL) was added iodomethane (11 μL, 0.18 mmol), and the mixture was stirred in the dark at rt for 3 d. The solid material was collected by filtration, washed with acetone, and dried in vacuo to give the product as a yellow powder (14.3 mg, 82%).HPLC 99%. 1H NMR (DMSO-d6) δ 2.80 (s, 3H), 3.25 (s, 3H), 4.43 (s, 3H), 7.38 (m, 1H), 7.62-7.68 (m, 2H), 8.35-8.43 (m, 3H), 9.95 (br s, 1H), 12.09 (s, 1H).

### Compound Example 8: 2-Hexyl-5,11-dimethyl-6H-pyrido[4,3-b]carbazol-2-ium iodide

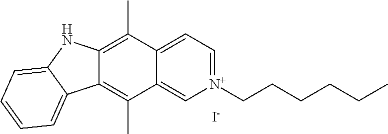 [SMILES: CCCCCC[n+]1ccc2c(C)c3[nH]c4ccccc4c3c(C)c2c1 ; [I-]]

To a suspension of 5,11-dimethyl-6H-pyrido[4,3-b]carbazole (9.2 mg, 0.037 mmol) in acetone (0.8 mL) was added (2-iodoethyl)benzene (22 μL, 0.15 mmol), and the mixture was heated by microwave irradiation at 100° C. for 1 h. The solid material was collected by filtration, washed with acetone, and dried in vacuo to give the product as a yellow powder (13.9 mg, 81%).HPLC 99%. 1H NMR (DMSO-d6) δ 0.86 (t, J=7.1 Hz, 3H), 1.24-1.40 (m, 6H), 2.01 (m, 2H), 2.85 (s, 3H), 3.32 (s, 3H), 4.70 (t, J=7.5 Hz, 2H), 7.40 (m, 1H), 7.62-7.69 (m, 2H), 8.45-8.49 (m, 2H), 8.53 (dd, J=7.2 Hz, J=1.1 Hz, 1H), 10.07 (d, J=0.8 Hz, 1H), 12.18 (s, 1H).

### Compound Example 9: 5,11-Dimethyl-2-(2-phenylethyl)-6H-pyrido[4,3-b]carbazol-2-ium iodide

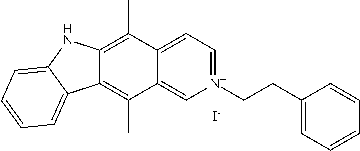 [SMILES: Cc1c2cc[n+](CCc3ccccc3)cc2c(C)c2c1[nH]c1ccccc12 ; [I-]]

To a suspension of 5,11-dimethyl-6H-pyrido[4,3-b]carbazole (9.6 mg, 0.039 mmol) in acetone (0.8 mL) was added 1-iodohexane (23 μL, 0.16 mmol), and the mixture was heated by microwave irradiation at 125° C. for 1 h. The solid material was collected by filtration, washed with acetone, and dried in vacuo to give the product as a yellow powder (15.7 mg, 84%).HPLC 95%, 95%. 1H NMR (DMSO-d6) δ 2.83 (s, 3H), 3.18 (s, 3H), 3.35 (t, J=7.4 Hz, 2H), 4.96 (t, J=7.4 Hz, 2H), 7.21-7.32 (m, 5H), 7.39 (m, 1H), 7.62-7.68 (m, 2H), 8.40-8.47 (m, 2H), 8.51 (dd, J=7.3 Hz, J=1.2 Hz, 1H), 9.81 (d, J=0.8 Hz, 1H), 12.17 (s, 1H).

### Compound Example 10: 2-(Carbamoylmethyl)-5,11-dimethyl-6H-pyrido[4,3-b]carbazol-2-ium iodide

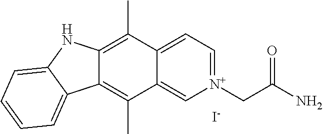 [SMILES: Cc1c2cc[n+](CC(N)=O)cc2c(C)c2c1[nH]c1ccccc12 ; [I-]]

To a suspension of 5,11-dimethyl-6H-pyrido[4,3-b]carbazole (9.5 mg, 0.039 mmol) in acetone (0.8 mL) was added 2-iodoacetamide (28.5 mg, 0.15 mmol), and the mixture was stirred at rt for 24 h. The solid material was collected by filtration, washed with acetone, and dried in vacuo to give the product as a yellow powder (12.4 mg, 75%).HPLC 99%. 1H NMR (DMSO-d6) δ 2.84 (s, 3H), 3.28 (s, 3H), 5.46 (s, 2H), 7.40 (m, 1H), 7.63-7.69 (m, 2H), 8.37 (dd, J=7.3 Hz, J=1.2 Hz, 1H), 8.41-8.47 (m, 2H), 10.01 (d, J=0.9 Hz, 1H), 12.19 (s, 1H).

### Prophetic Compound Examples

Furthermore, the following compounds are prepared in accordance with the invention (in the presence of suitable anions):

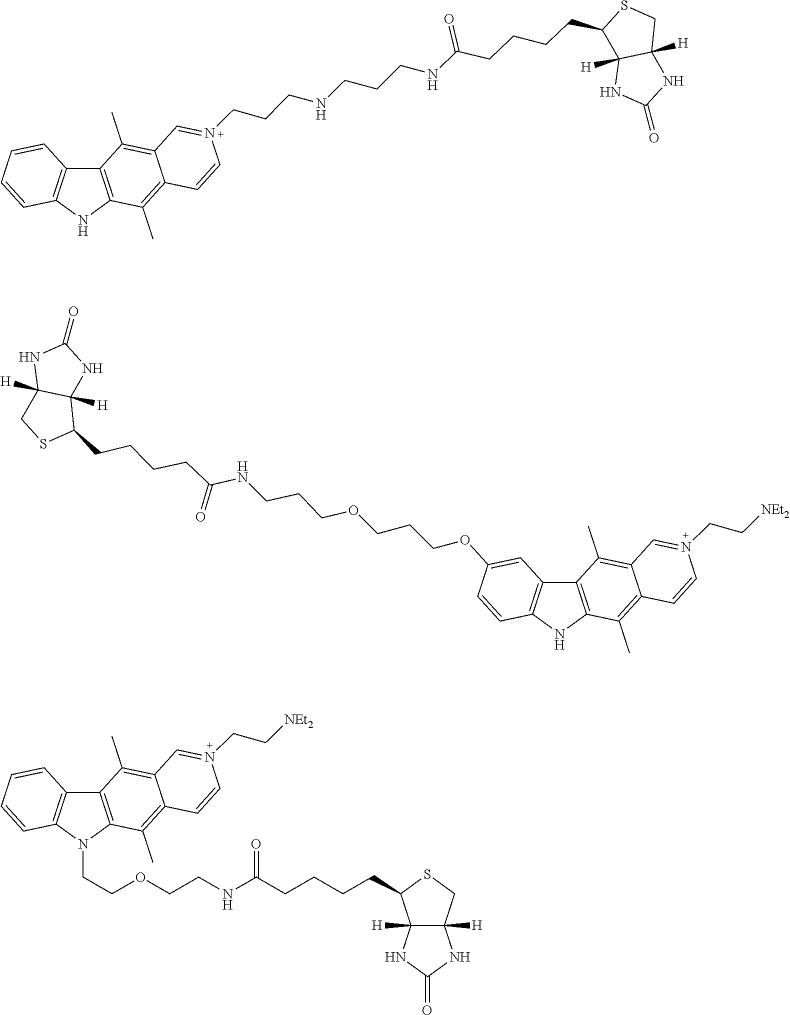

### Biological Examples

Cell culture and transfection: HeLa, MCF7, MDA-MB-231, U20S, HCT116, SK-N-F1, SK-N-RA, SK-N-AS, COS-7, Rat1 fibroblasts (Tgrl, H015.19 and HOMYC3), primary rat embryonic fibroblasts (REFs) were maintained in DMEM and Kelly, IMR-32, SK-N-DZ, P493-6, Daudi, CA46 and Ramos were kept in RPMI. In addition, the medium for H015.19 and primary REFs contained 1% sodium pyruvate. All cells were mycoplasma free and kept at 37° C. and 5% CO₂. U20S-MYC-ER cell lines were cultured in phenol-red free DMEM and treated with 100 nM 4-hydroxytamoxifen (4-OHT) (Sigma-Aldrich) to activate MYC-ER. To turn off expression of the MYC gene in P493-6 cells, a final concentration of 1 μg/ml doxycycline (Sigma) was added to the culture medium for 24 hours (Schuhmacher M, et al. (1999) Control of cell growth by c-Myc in the absence of cell division. Curr Biol 9(21):1255-1258, the contents of which are incorporated herein by reference). Transfection of HeLa cells was performed using Lipofectamine 2000 reagent (Invitrogen) according to manufacturer's instructions. For transfection of 293-T, U20S and REFs, FuGene6 (ROCHE) was used in accordance with manufacturer's recommendations. The proteasome inhibitor Z-leu-leu-leu-H aldehyde (MG115) (Peptides International) was used at 40 μM concentration.

For anchorage-independent growth assay, cells were suspended in 250 μl 0.35% SeaPlaque agarose (InVitro) and seeded into 24 well plates which had previously been coated with a bottom layer of 250 μl 0.7% agarose. After 16 days, the colonies were stained with 100 μg/ml MTT (Sigma) overnight and colonies were counted. For foci formation assays, 3×10⁴ REFs were seeded into 6 well plates. The following day the cells were transfected with the indicated plasmids (0.3 μg of each, a total of 0.6 μg per well). After another 24 hours compounds were added. The medium was changed every 2-3 days. When the cells had grown into confluency, medium containing 4% serum was added. After 14 days the number of foci was scored.

Cell growth and viability assays: Cell growth and viability was estimated in triplicates with WST-1 (Roche) or Resazurin sodium salt (Sigma-Aldrich) assays in medium at 37° C. and 5% CO₂ for 2 hours after which absorbance or fluorescence, respectively, was measured with an Omega Fluostar (BMG Labtech) in a 96 well plate format. Cell viability by the Cyt60 assay was carried out as in manufacturers' protocol. For apoptosis assays, cells were seeded into 96 well plates, treated with compounds, and 24 hours later the cells were harvested and 2500 cells per analysis were used in the Cell Death Detection ELISA^{plus} kit (ROCHE) according to the manufacturer's recommendations. To evaluate cell viability in combination with apoptosis (caspase activation), the Apo-Tox Glo Triplex assay (Promega) was used. Fluorescence detection of viability (400Ex/505Em) and luminescence measurement of caspase activation was obtained in a 96-well plate format., according to the manufacturer's instructions.

Viability measurements in glioblastoma initiating cell cultures: The adherent human glioblastoma initiating cell cultures (2) were seeded on laminin-coated 384-well microplates (BD Falcon Optilux #353962). The optimal cell number for each culture was determined to ensure that it was in growth phase at the end of the assay (˜70% confluency which resulted in between 2000-4000 cells/well). Cell cultures (45 for screen 2 and 24/29 for screen 3) were plated one day prior to treatment using a Multidrop plate dispenser (ThermoScientific). Drugs were transferred using an ECHO550 non-contact liquid dispenser (Labcyte, USA) to a 384 standard dispensing plate. The substances were subsequently diluted in medium and lifted with a 384 head and a Janus MTD liquid dispenser (PerkinElmer, USA) to the cell plates. The screening experiment was assayed for viability using resazurin (R7017, Sigma Aldrich) after 72 hours of treatment as previously described (3), and detected by a fluorescent plate reader (EnVision multilabel reader, PerkinElmer) at excitation and emission wavelengths of 535/595 nm. As a negative control, DMSO vehicle was added at 4 different concentrations (16 wells/plate) and doxorubicin was used as a positive control (one 11-dose response curve on each plate). Each treatment dose was mapped to their individual exact DMSO concentration for normalization. The screening protocol included a dose range of 11 doses ranging from 20 nM-100 μM (screen 2) and 0.5 nM-50 μM (screen 3). All cell cultures are profiled and mapped to a specific glioma subgroup (Voichita Marinescu and Sven Nelander, unpublished), included in the analysis pipeline. Cells were screened in batches of 6-12 in an effort to optimize mixing of subgroups.

Plasmids: Zip-hGLuc(1) and Zip-hGLuc(2) kindly provided by S. Michnick (University of Montreal, Montreal) were used to create MYC-Luc2, NMYC-Luc2 and MAX-Luc1 by replacing the existing Zip-gene (GCN4) with full length MYC, MYCN or MAX cDNA. Vectors where the Zip-protein was cut out but not replaced (Luc1 and Luc2) was also created (details available on request). Other plasmids used were pCMV-LUC, pLXSMYCN, pEVBJ-RAS, pCDNA3-flag-MYC wt, T58A, S62A, and pCDNA3. N-terminal 6xHis-tagged constructs MYCbHLHZip and MAXbHLHZip were cloned into pET28a.

In situ proximityligation assay (isPLA): The isPLA assay has been described previously (Castell A, et al. (2018) A selective high affinity MYC-binding compound inhibits MYC:MAX interaction and MYC-dependent tumor cell proliferation. Sci Rep 8(1):10064′; the contents of which are incorporated herein by reference). Briefly, cells were grown on collagen-coated chamber slides (Falcon), treated with compounds and then washed twice with PBS and fixed in ice cold methanol for 5-15 min at room temperature. Slides were washed in PBS with 0.05% Tween 20 and incubated in blocking buffer after which isPLA was performed using the Duolink® in situ PLA kit (Sigma-Aldrich) according to the manufacturer's protocol. DNA was stained with DAPI. Incubation with primary antibodies were performed at +4° C. overnight. Images were taken using an Axiovert 200M inverted microscope (Zeiss) and fluorescent dots were quantified using semi-automated analysis in ImageJ (http://imagej.net) and averaged to number of dots per cell. Antibodies used are listed below.

Western blot, co-immunoprecipitation, chromatin immunoprecipitation cycloheximide chase: Western blot, immunoprecipitations and cycloheximide chase were performed essentially as described in (Bahram F, Wu S, Oberg F, Luscher B, & Larsson L G (1999) Posttranslational regulation of Myc function in response to phorbol ester/interferon-gamma-induced differentiation of v-Myc-transformed U-937 monoblasts. Blood 93(11):3900-3912; the contents of which are incorporated herein by reference). For western blot equal amounts of proteins (30-50 g) were separated on a 4-12% SDS-PAGE gel. Proteins were transferred to Immobilion-P membrane (Millipore) and detected by immunostaining and chemoluminescence (Immobilion western HRP substrate; Millipore). For immunoprecipitations 500-1000 μg of protein was used. Chromatin immunoprecipitation were performed as described (Bahram F, et al. (2016) Interferon-gamma-induced p27KIP1 binds to and targets MYC for proteasome-mediated degradation. Oncotarget 7(3):2837-2854; the contents of which are incorporated herein by reference). Briefly, cells were crosslinked with 1% formaldehyde on ice for 6 minutes. Nuclear chromatin was sonicated on ice to fragments from 0.3 kb to 0.5 kb. Nuclear chromatin equivalent to 2.5×10⁷ cells was immunoprecipitated with 2 μg antibody.

Antibodies: Antibodies used for isPLA for cell cultures were C-33 α-MYC or B8.4.B α-MYCN combined with C-17 α-MAX or H-50 α-FRA1 (all Santa Cruz Biotechnology) combined with 2315S α-JUN (Cell Signaling Technology), or control DBD α-Gal4 antibody (Santa Cruz), all diluted 1:50. Immunoprecipitation of MYC was performed with α-MYC N262 (Santa Cruz biotechnology) or α-MYC Y69 (Abcam) antibodies. For ChIP, α-MYC N262 (Santa Cruz biotechnology) was used. The following antibodies were used for western blot: α-MYC N262, α-MYC 9E10, α-MYC C-33, α-NMYC (d46-507), α-Cyclin E HE12 (all Santa Cruz), α-MYC Y69 (Abcam), α-P-T58/S62 MYC (Cell Signaling), α-MAX 101271 (Abcam), α-Actin (A2228; Sigma-Aldrich or AC-15, Sigma). For staining of tumor tissue sections, the Ki67 Ab 16667 (Abcam, dilution 1/500), the CD31 Ab 553370 (BD Pharmingen, dilution 1/200), MYC Ab Y69 (Abcam, dilution 1/250), MYCN Ab B8.4.B (Santa Cruz biotechnology, dilution 1/250), Casp3 Ab D175 (Cell Signaling, dilution 1/250) were used.

Primers for RT-qPCR: The following forward (FW) and reverse (REV) sequences of human primers were used for RT-qPCR:
GAPDH FW: ACATCGCTCAGACACCATG, REV: TGTAGTTGAGGTCAATGAAGGG, ODC1 FW: TCTGCTTGATATTGGCGGTG, REV: GGCTCAGCTATGATTCTCACTC, CR2 FW: GGGTTTTCTTGGCTCTCGTC, REV: CCTTATCACGGTACCAACAGC, RGS16 FW: CTGCGATACTGGGAGTACTGG, REV: CCACCCCAGCACATCTTC CAMKV FW: TGGCTGGTGACTATGAGTTTG, REV: CAGCATTGCCAGAAATCCAC Nucelolin FW: AGGTGACCCCAAGAAAATGG, REV: AGCCTTCTTGCCTTTCTTCTG

Recombinant proteins: Recombinant proteins containing His-tagged N-termini were over-expressed in E. coli BL21 (DE3) bacteria (Stratagene) at 37° C. in 2XTY, or LB, media with kanamycin, and were purified on a Ni-NTA (Qiagen) affinity bench column, or using a HisTrap HP column (5 mL) with an AKTA system. The purifications were carried out according to manufacturer instructions. All the proteins were dialyzed against PBS, pH 7, at 5° C. overnight. The purity of the proteins was confirmed by SDS-PAGE analysis and Mass Spectrometry analysis.

Gaussia luciferase protein fragment complementation assay (GLuc): The Gaussia luciferase protein fragment complementation assay has been described elsewhere (Castell A, et al. (2018) A selective high affinity MYC-binding compound inhibits MYC:MAX interaction and MYC-dependent tumor cell proliferation. Sci Rep 8(1):10064; Remy I & Michnick S W (2006) A highly sensitive protein-protein interaction assay based on Gaussia luciferase. Nat Methods 3(12):977-979; the contents of each of which are incorporated herein by reference). 0.2-0.4 μg of each GLuc-construct together with 0.05 μg pCMV-LUC (firefly luciferase) were used per transfection in 12 well plates. 24 hours later cells were treated with compound or DMSO. After another 17 hours, the cells were harvested and lyzed in passive lysis buffer (Promega) supplemented with complete protease inhibitor (Roche). After 60 min incubation at room temperature 20 μM D-luciferin was added (substrate of Firefly luciferase) and luminescence was measured in a Lumat LB9501 (Berthold) or OmegaFluostar (BMG Labtech) luminometer. Directly after, Gaussia luciferase substrate Coelenterazine (Promega) was added to a final concentration of 20 μM in a mixture with “Stop n' Glow” (Promega) and the luciferase activity was measured. The ratio between Gaussia and Firefly luciferase values were calculated and normalized to DMSO-treated control cells.

Surface plasmon resonance (SPR): The SPR experiments were performed at 25° C. using a Biacore T200 (GE Healthcare) instrument kindly provided by SciLifeLab Solna. An amino coupling procedure was used to immobilize protein on a CM5 sensor chip (GE Healthcare). Sensorgrams were generated by subtraction of the reference (blank immobilized) surface. The MYC SPR assay was carried out as described above with MYCbHLHZip immobilized to a level of 800-1000 RU. For kinetic binding experiments, a Langmuir 1:1 binding event was applied using the Biacore T200 Evaluation Software 2.0 (GE Healthcare) to determine association (kₐ) and dissociation (k_{d}) constants of the compound and to calculate affinity (K_{D}) by the formula; K=k_{d}/kₐ. Binding responses from equilibrium binding experiments were plotted against compound concentration and K_{D} values were determined at 50% of the theoretical Rmax with the formula Rmax=(MW analyte/MW ligand)×immobilized ligand level on the chip (RU)×stoichiometry (1:1). Same coupling method described above was used to immobilize the control proteins. MAX was immobilized to approximately 2000 RU.

Flow cytometry: P493-6 cells were synchronized using 1 μg/ml doxycycline. The cells were washed three times with culture medium and incubated with or without compound for another 48 hours before harvest. REFs were seeded out in 6 well plates 24 hours prior to indicate treatment and harvested by trypsinization after 48 hours of treatment. Cells were stained with propidium iodide (Sigma) and 1×10⁴ cells per treatment were analyzed using a FACScan and CellQuestPro (Becton Dickinson). The cell cycle distribution analysis was performed using the ModFit LT 3.1 software (Verity software House).

Top2 decatenation assay: Recombinant human Top2a (15 nM) was incubated with 80 ng catenated kinetoplast DNA (Topogen, TG2013) in Top2 reaction buffer (50 mM Tris pH8, 150 mM NaCl, 10 mM MgCl₂, 0.5 mM DTT, 30 μg/ml BSA) with 1-100 μM of either doxorubicin, MYCMI-7, ellipticine or camptothecin for 10 minutes at 37° C. Drug dilutions were made in a DMSO/H₂O solution so that a final concentration of 1% DMSO was consistent across all samples. The reaction was stopped with the addition of SDS to a final concentration of 1% and the Top2a was digested by Proteinase K at 55° C. for 10 minutes. DNA was purified by phenol:chloroform extraction before running on a 1% agarose gel either containing or post-stained with 0.5 μg/ml ethidium bromide. The decatenated products were quantified, normalised to the Top2α+DMSO control and plotted using GraphPad Prism.

Pharmacokinetics: MYCMI-7 was dissolved to 12.5 mg/mL in DMSO:glycerol 9:1 and was further diluted to 1.25 mg/mL in 9% DMSO:saline before administration to mice by intraperitoneal (i.p.) injection. Blood and brains were collected from three to four mice per group at 0, 1, 2, 4 and 24 hours following injection of 6.25 mg/kg ToM. Plasma samples were extracted using protein precipitation. Brain samples were extracted by homogenization in acetonitrile:DMSO, 95:5 using a gentleMACS Dissociater and gentleMACS™ M Tubes. The protein in the mouse plasma samples was removed by protein precipitation with acetonitrile containing the internal standard. After protein precipitation the study samples were centrifuged, and the resulting supernatant was analyzed. The acetonitrile:DMSO from the brain sample homogenates were spiked with the internal standard and then analyzed. All samples were analyzed by first separating them by reversed phase gradient LC and subsequently detecting them using positive electrospray ionization and multiple reaction monitoring (MRM).

RNA-seq analysis: Libraries for RNA sequencing were prepared using the TruSeq Stranded Total RNA kit with RiboZero (Illumina) and sequenced on two lanes of the HiSeq 2500 platform with a single-end 1×51 setup and the HiSeq Rapid SBS v2 chemistry. Demultiplexed .fastq files were aligned to the human GRCh37 reference genome using Tophat v 2.0. After alignment, bam files from two separate flowcell lanes were merged using samtools. Raw read counts per gene were then generated using htseq-count v0.6.1. Differential expression analysis comparing the two DMSO-treated to the two MYCMI-7 treated samples was performed using the R/Bioconductor DESeq2 package v1.26.0 (Bioconductor v3.10, R v3.6.1), Following differential expression analysis, all genes were ranked according to adjusted p value and log fold change. Gene set enrichment analysis was then performed using GSEA software with the Hallmarks (H) and curated (C2:CGP) MSigDB gene sets; v6.2.

Mouse tumor models: All animal protocols in these studies were approved by the ethical committee for animal experiments of northern Stockholm (N47/14, N241/15 and N231/14) and of Uppsala (C41/14). Mice were maintained under pathogen-free conditions according to guidelines of the animal facility at MTC, Karolinska Institutet or at AKM, Karolinska University Hospital.

For the AML tumor model, MYC+BCL-XL expressing, GFP+ leukemic cells were isolated from cells leukemic mice as described (Bazzar W, et al. (2021) Pharmacological inactivation of CDK2 inhibits MYC/BCL-XL-driven leukemia in vivo through induction of cellular senescence. Cell Cycle 20(1):23-38; the contents of which are incorporated herein by reference). 3×10⁵ cells were injected into each recipient C57BL mouse after irradiation (600 rad). AML-like leukemia initiation was confirmed via flow cytometry and Giemsa staining, at day 8 after transplantation. MYCMI-7 were then administrated intraperitoneally daily at a dose of 12.5 mg/kg. Liver, spleen and bone marrow were extracted at day 11, 15 and endpoint for measurement. In addition, body weight of mouse in each group was also collected every 4^{th} day until the endpoint of the experiment.

For the xenograft transplantation mouse tumor models of breast cancer and neuroblastoma, 6-8 weeks old NOD/SCID mice (Taconic) or NMRI nu/nu (Scanbur) were injected s.c. with 5×10⁶ MDA-MB-231 breast cancer cells or MYCN-amplified SK-N-DZ neuroblastoma cells. When tumors reached a size of 100-200 mm³, mice started receiving treatment (6.25 mg/kg MYCMI-7) administered intratumorally (50 ml) twice per week. The last dose was administered 3 hrs before termination. Mice were sacrificed when the tumors had reached as size of 1000 mm³ (MDA-MB-231) or 1500 mm³ (SK-N-DZ). Tumors were collected and frozen in OCT (Cryomount, Histolab) or fixed in buffered 4% formaldehyde solution (Histolab) and paraffin-embedded.

For description of immunohistochemistry and immunofluorescence see below.

Immunohistochemistry andimmunofluorescence: Apoptosis was visualized at the single-cell level on tumor cryosections using the TUNEL method using the In Situ Cell Death Detection Fluorescein-Roche kit and analyzed by fluorescence microscopy. Briefly, tumor cryosections were fixed in 4% paraformaldyde (PFA) and the assay was performed following the instructions from the manufacturer. Negative controls were run using the reagent without the TdT enzyme. Samples were mounted using Vectashield mounting medium (Vector) and DAPI was used as nuclear counterstaining. Cell proliferation and microvascular density (MVD) were evaluated through Ki67 and CD31 staining, respectively, on tumor cryosections and detected by immunofluoresence. Visualization and image acquisition was done in a Zeiss microscope, and for panoramic views by a Vectra imaging system. For quantification, areas with positive TUNEL and Ki67 staining were measured using Image J software. For the analysis of MVD, the number of vessel structures per microscopic field was calculated.

Statistical analysis: Proportional data corresponding to GLuc, isPLA in cells for MYC:MAX and qPCR experiments in U20S cell line were analyzed with a non-parametric Kruskal-Wallis test, while comparisons across treatments were performed with Bonferroni post hoc or Wilcoxon tests, to account for heteroscedasticity. Proportional data corresponding to MYCN:MAX isPLA and RT-qPCR experiments in neuroblastoma cell lines were initially transformed (square root and arcsine) and analyzed with generalized linear models (GLMs) with a normal distribution. Pairwise comparisons were performed with Bonferroni post hoc tests. Proportional data corresponding to isPLA in vivo experiments were log-transformed and analyzed with a GLM. The effect of MYC mRNA and protein levels on the response of the NCI-60 cancer cell lines to MYCMI-6 were analyzed with GLM assuming a normal distribution, while the hypothesis on the probability of a cell line with “high MYC” or “low MYC” protein levels to respond to MYCMI-6 was tested with the binomial exact test. The rest of the data were analyzed with the Student's t-test. All analyses were carried out in R (v. 3.3.3; R Foundation for Statistical Computing, Vienna, AT), at a level of significance α=0.05, with packages car, agricolae, multcomp and MASS.

Survival in the animal studies was evaluated with log rank test.

### Biological Example 1: MYCMI-7 Inhibits the MYC:MAX Interaction in Cells, Binds MYC In Vitro, and Blocks MYC Function

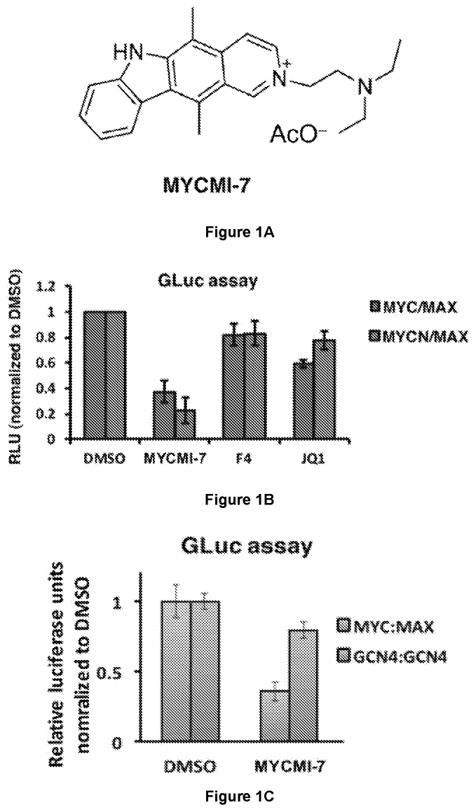

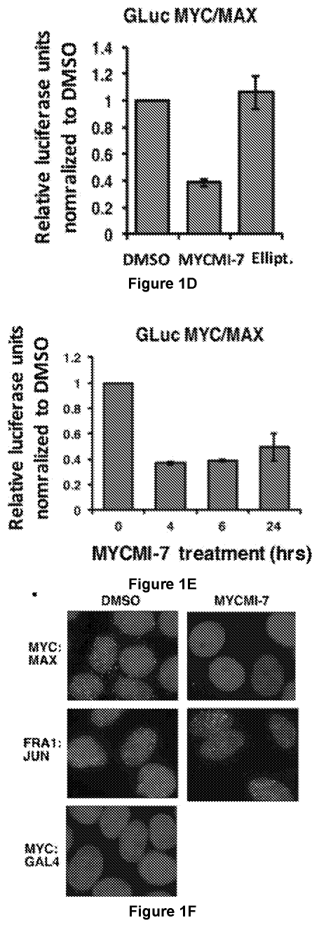

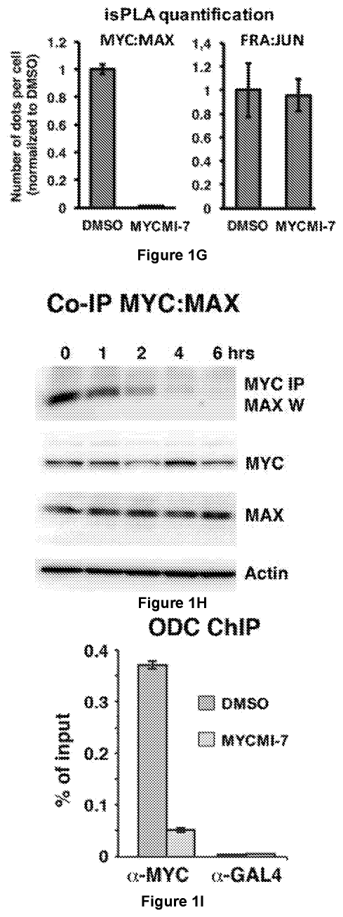

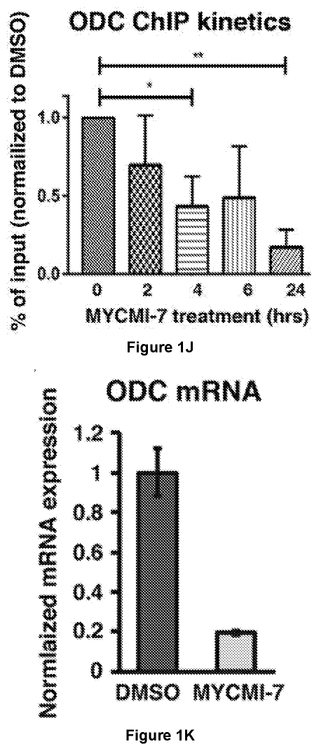

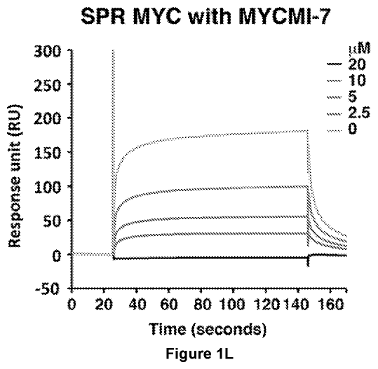

To verify that MYCMI-7 inhibits MYC:MAX dimerization in cells, we utilized the split Gaussia princeps luciferase (GLuc) assay, with GLuc fragments fused to MYC (or MYCN) and MAX, respectively (Castell A, et al. (2018) A selective high affinity MYC-binding compound inhibits MYC:MAX interaction and MYC-dependent tumor cell proliferation. Sci Rep 8(1):10064; Remy I & Michnick S W (2006) A highly sensitive protein-protein interaction assay based on Gaussia luciferase. Nat Methods 3(12):977-979; the contents of each of which are incorporated herein by reference). MYCMI-7 (5 μM) treatment inhibited both the MYC:MAX and MYCN:MAX Gluc interactions, whereas previously published MYC:MAX inhibitor 10058-F4 (64 μM) (Yin X, Giap C, Lazo J S, & Prochownik E V (2003) Low molecular weight inhibitors of Myc-Max interaction and function. Oncogene 22(40):6151-6159; the contents of which are incorporated herein by reference) and the bromodomain inhibitor JQ1 (5 μM) (Filippakopoulos P, et al. (2010) Selective inhibition of BET bromodomains. Nature 468(7327):1067-1073; the contents of which are incorporated herein by reference) had only weak effects (FIG. 1B). In contrast, MYCMI-7 did not have any significant effect on homodimerization of the bZip protein GCN4 (FIG. 1C). Although MYCMI-7 shows structural similarities to ellipticine, the latter was inactive at the same concentration (FIG. 1D), and was hereafter used as a reference compound. The effect of MYCMI-7 was rapid; MYC:MAX interaction was inhibited already within 4 hours of treatment and thereafter (FIG. 1E). To validate the effect of MYCMI-7 on endogenous MYC:MAX interactions, in situ proximity ligation assay (isPLA) (Castell A, et al. (2018) A selective high affinity MYC-binding compound inhibits MYC:MAX interaction and MYC-dependent tumor cell proliferation. Sci Rep 8(1):10064; Soderberg O, et al. (2006) Direct observation of individual endogenous protein complexes in situ by proximity ligation. Nat Methods 3(12):995-1000; the contents of each of which are incorporated herein by reference) was used. 5 μM MYCMI-7 strongly inhibited the MYC:MAX interaction in MCF-7 breast cancer cells, but not the interaction between the bZip proteins FRA1 and JUN (FIG. 1F-G). In addition, co-immunoprecipitation (co-IP) showed that the MYC:MAX interaction decreased already at 1 hour after treatment, and was reduced drastically at 4 hours, without having much effect on the total level of MYC at these time points (FIG. 1H).

Further, chromatin immunoprecipitation (ChIP) in breast cancer cells showed that association of MYC with the ODC1 gene promoter, a well-known MYC target gene, was reduced by MYCMI-7 treatment (5 μM), starting already after 2 hours and reaching a minimal level from 4 hours, coinciding with reduced ODC1 expression (FIG. 1I-K).

To further investigate potential direct binding of MYCMI-7 to MYC, an equilibrium surface plasmon resonance (SPR) assay was carried out using the recombinant bHLHZip domain of the MYC protein. A dose-dependent increase in MYCMI-7 binding was observed, with association and dissociation curves indicating that MYCMI-7 binds directly to MYC with a K_{D} of approximately 4 μM as determined by Langmuir 1:1 analysis (FIG. 1L).

We concluded that MYCMI-7 binds MYC in vitro, and rapidly, strongly and selectively inhibits MYC:MAX interaction and MYC's association with chromatin in cells at low M concentrations.

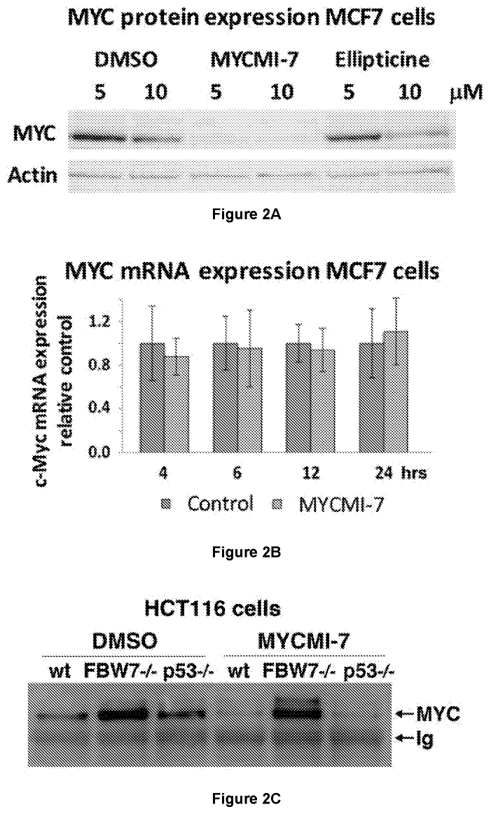

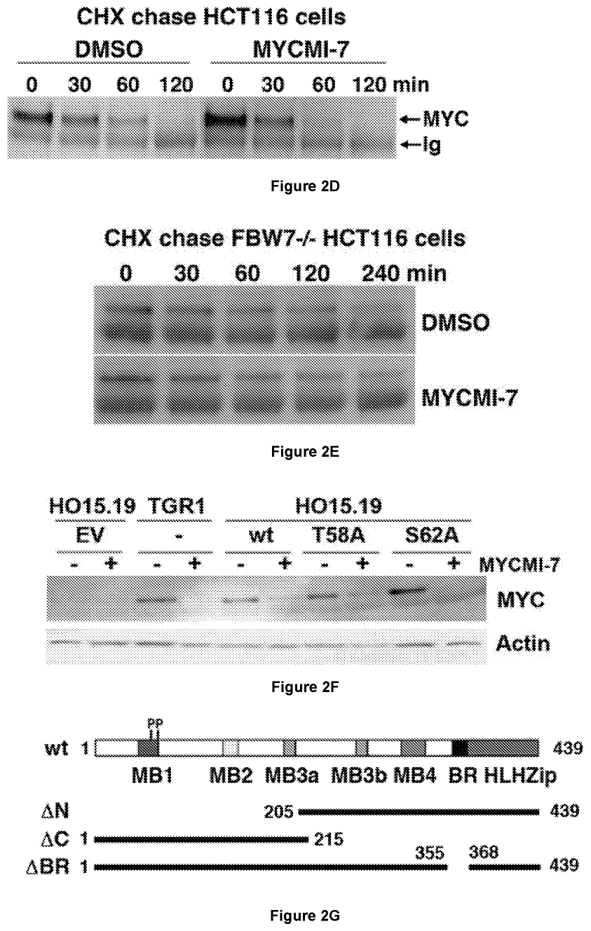

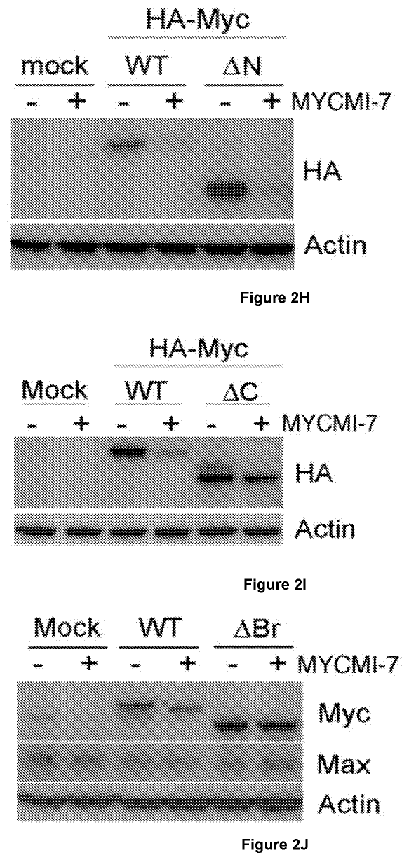

Biological Example 2: MYCMI-7 increases MYC protein turnover We next studied the expression levels of MYC and MAX after MYCMI-7 treatment. The steady state level of MYC protein decreased drastically in MCF7 cells after 17 hrs treatment with 5 μM MYCMI-7, but not after treatment with ellipticine (FIG. 2A). This effect was confirmed in HeLa, P493-6 and HCT116 cells. Expression of MYCN was also reduced in Kelly neuroblastoma cells (data not shown). The decrease was not due to reduced MYC mRNA expression as determined by RT-qPCR (FIG. 2B). We therefore looked at MYC protein stability using HCT116 cells deficient in FBXW7, the main E3 ubiquitin ligase targeting MYC (Welcker M, et al. (2004) The Fbw7 tumor suppressor regulates glycogen synthase kinase 3 phosphorylation-dependent c-Myc protein degradation. Proc Natl Acad Sci USA 101(24):9085-9090; Yada M, et al. (2004) Phosphorylation-dependent degradation of c-Myc is mediated by the F-box protein Fbw7. Embo J 23(10):2116-2125; the contents of each of which are incorporated herein by reference). Interestingly, MYC protein level was unaffected in the FBXW7−/− cells, while strongly reduced in wt and p53 null cells after MYCMI-7 exposure (FIG. 2C). Further, cycloheximide (CHX) chase assay showed that the turnover rate of MYC was increased in wt HCT116 cells but not in FBXW7−/− HTC116 cells in response to MYCMI-7 (FIG. 2D and E), suggesting that MYCMI-7 induces degradation of MYC in a FBXW7-dependent manner. However, protein expression of cyclin E, another FBXW7 target, was unaffected (data not shown), excluding a general increase in FBXW7 activity upon MYCMI-7 treatment. Further, phosphorylation of MYC at Thr-58 or Ser-62, which are binding sites for FBXW7, did not increase in response to MYCMI-7 (data not shown). To further investigate this, H015.19 MYC knockout Rat1 cells were reconstituted with T58A or S62A MYC mutants, or wt MYC as control. MYCMI-7 treatment reduced the levels of both wt and the MYC mutants (FIG. 2F). Similar results were obtained in U2OS cells transiently transfected with vectors expressing wt MYC and T58A or S62A MYC mutants (data not shown).

To map regions of MYC involved in MYCMI-7-mediated MYC turnover, different MYC mutants were utilized in transient transfection assays (FIG. 2G). This analysis showed that the C-terminus, but not the N-terminus, of MYC was required for turnover. Further mapping of the C-terminal part showed that the DNA-binding basic region was required for MYCMI-7-mediated MYC turnover (FIG. 2G-J).

### Biological Example 3: MYCMI-7 Reduces Tumor Cell Growth and Viability in a MYC-Dependent Manner and Down-Regulates the MYC Pathway

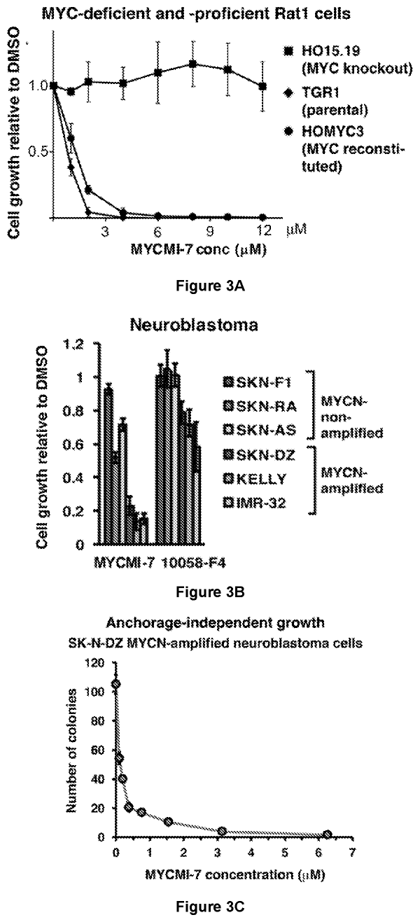

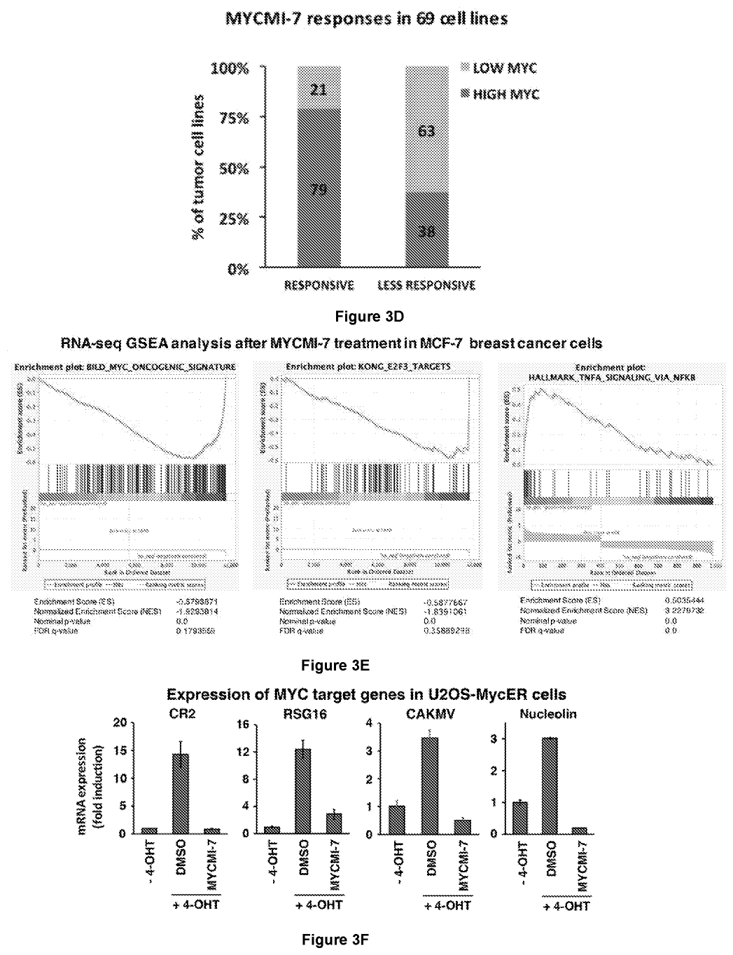

To address whether MYCMI-7 affects cell growth in a MYC-dependent manner, we utilized immortalized Rat1 fibroblasts with different MYC status; H015.19 are MYC null cells derived from TGR1 (parental cell line) and HOMyc3, which are HO15.19 cells reconstituted with MYC (Mateyak M K, Obaya A J, Adachi S, & Sedivy J M (1997) Phenotypes of c-Myc-deficient rat fibroblasts isolated by targeted homologous recombination. Cell Growth Differ 8(10):1039-1048; the contents of which are incorporated herein by reference). Proliferation/viability of cells expressing MYC declined drastically at low concentrations of MYCMI-7, with an average growth inhibition of 50% (GI₅₀) around 2 μM as measured by WST-1 assay (which measures metabolic activity in cells), while the MYC null cells were unaffected even at concentrations of 10 μM (FIG. 3A), demonstrating that the effect of MYCMI-7 was MYC-dependent. In contrast, ellipticine reduced growth of all three cell clones, irrespective of MYC status (data not shown). When H015.19 cells were exposed to MYCMI-7 concentrations higher than 10 μM, viability started to go down, suggesting that off target effects started to appear above this concentration (data not shown).

To investigate if MYCMI-7 affects growth of typical MYC-driven tumor cells, we utilized a panel of childhood neuroblastoma cells with or without MYCN-amplification as well as a set of Burkitt's lymphoma cell lines, which carry MYC translocations. Treatment with MYCMI-7 reduced tumor growth and viability in all the neuroblastoma cell lines, but the effect was significantly stronger in the MYCN-amplified cases (FIG. 3B). Note that non-MYCN-amplified neuroblastoma cells express MYC, albeit at a lower level than MYCN in amplified lines (Castell A, et al. (2018) A selective high affinity MYC-binding compound inhibits MYC:MAX interaction and MYC-dependent tumor cell proliferation. Sci Rep 8(1):10064; the contents of which are incorporated herein by reference). The efficiency of MYCMI-7 towards MYCN-amplified neuroblastoma cells was even stronger in 3D cultures, with GI₅₀ in the nanomolar range (FIG. 3C). MYCMI-7 also strongly reduced growth of the three Burkitt's lymphoma cell lines (data not shown).

To investigate whether the levels of MYC expression in tumor cells correlate with growth inhibitory response to MYCMI-7, we utilized GI₅₀ and mRNA expression data from the NCI-60 diverse human tumor cell line panel available for MYCMI-7 by the Developmental Therapeutics Program of the U.S. National Cancer Institute (DTP-NCI). The data was extracted from the NIH-supported CellMiner™ version 2.1 (https://discover.nci.nih.gov/cellminer) and combined with MYC protein data obtained from Novartis proteome scout SymAtlas Project (https://proteomescout.wustl.edu/proteins/52581/expression) or elsewhere in the literature as described previously (Castell A, et al. (2018) A selective high affinity MYC-binding compound inhibits MYC:MAX interaction and MYC-dependent tumor cell proliferation. Sci Rep 8(1):10064; the contents of which are incorporated herein by reference). The cell lines were categorized as “responsive” or “less responsive” to MYCMI-7 based on average logarithmic GI₅₀ values, as well as the categories “higher MYC” and “lower MYC” based on higher or lower than average MYC mRNA and/or high protein levels. There was a significant correlation between the response to MYCMI-7 and the MYC mRNA/protein levels among the 60 tumor cell lines (FIG. 3D). 79% of the cells with high MYC mRNA/protein level were responsive to MYCMI-7 and 63% of the cells with low MYC levels were less responsive (FIG. 3D). This indicates that cells with high MYC levels are more likely to respond to MYCMI-7 treatment than cells with low MYC levels.

To investigate whether MYCMI-7 could inhibit MYC-induced oncogenic transformation of normal cells together with H-RAS, normal rat embryonic fibroblasts (REFs) were transfected with MYC+RAS vectors. Formation of transformed foci as well as the ability of MYC+RAS-transformed REFs to form colonies in semi-solid medium was strongly inhibited by treatment with MYCMI-7 (data not shown).

To study the impact of MYCMI-7 on global gene expression, we next performed RNA-seq analysis in MCF-7 breast carcinoma cells after treatment with 5 μM MYCMI-7 for 24 hrs. Gene set enrichment analysis (GSEA) of differentially expressed genes showed a downregulation of the MYC and E2F target genes (FIG. 3E, left and middle panels). In contrast, upregulated genes were enriched in pathways associated with inflammatory signaling via NFkB, which is consistent with the reported suppressive action of MYC on immune signaling (FIG. 3E, right panel) (Casey SC, et al. (2016) MYC regulates the antitumor immune response through CD47 and PD-L1. Science 352(6282):227-231; Kortlever R M, et al. (2017) Myc Cooperates with Ras by Programming Inflammation and Immune Suppression. Cell 171(6):1301-1315 e1314; the contents of each of which are incorporated herein by reference). To further document the impact of MYCMI-7 on MYC's transcriptional activity, we utilized U2OS cells expressing a MYC-estrogen receptor (MYCER) fusion protein, which is regulated by 4-hydroxytamoxifen (4-OHT) (Adhikary S & Eilers M (2005) Transcriptional regulation and transformation by Myc proteins. Nat Rev Mol Cell Biol 6(8):635-645; the contents of which are incorporated herein by reference). Treatment with MYCMI-7 significantly reduced 4-OHT-induced expression of CR2, RGS16, CAMKV and nucleolin (FIG. 3F), which all previously has been characterized as direct MYC target genes (Sabo A, et al. (2014) Selective transcriptional regulation by Myc in cellular growth control and lymphomagenesis. Nature 511(7510):488-492; Walz S, et al. (2014) Activation and repression by oncogenic MYC shape tumour-specific gene expression profiles. Nature 511(7510):483-487; the contents of each of which are incorporated herein by reference).

Taken together, these results suggest that MYCMI-7 reduces tumor cell growth/viability in a MYC-dependent manner and down-regulates the MYC pathway.

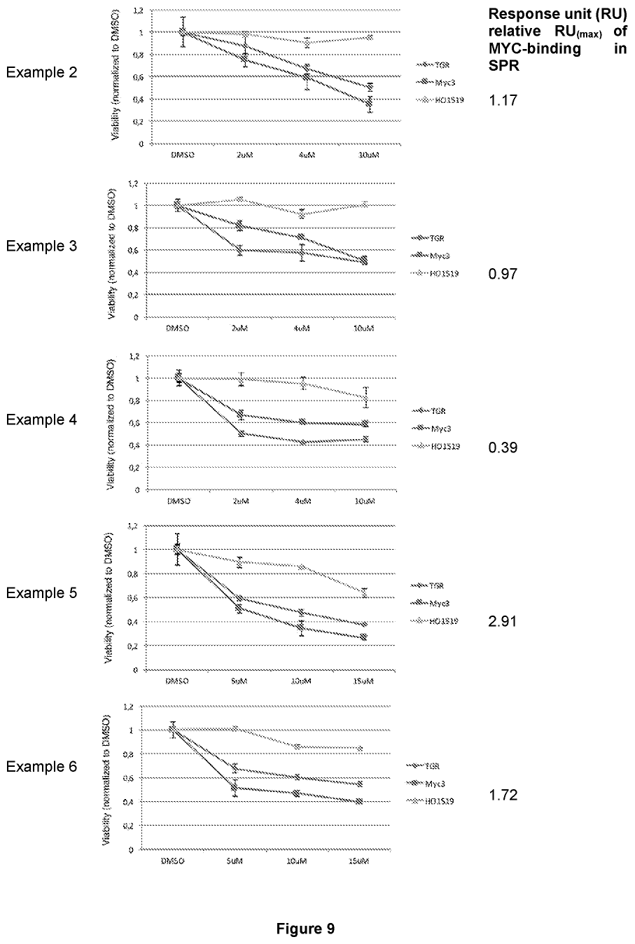

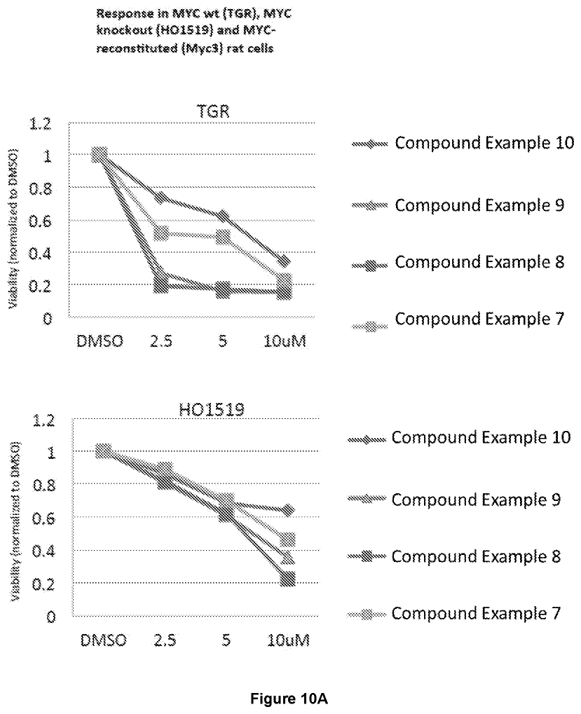

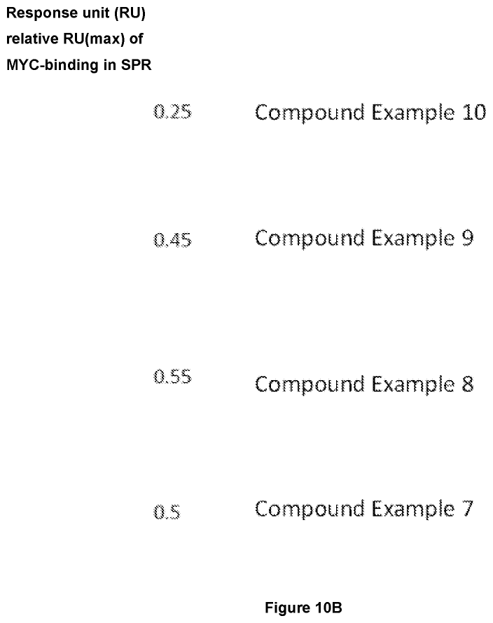

Experiments to determine whether compounds affect cell growth in a MYC-dependent manner were repeated using the same techniques for the compounds of Examples 2 to 6 in MYC wt (TGR), MYC knockout (HO15.19) and MYC-reconstituted (Myc3) rat cells. The results are as shown in FIG. 9.

### Biological Example 4: MYCMI-7 Induces Growth Arrest and Apoptosis in Malignant Cells and Only Growth Arrest in Normal Cells

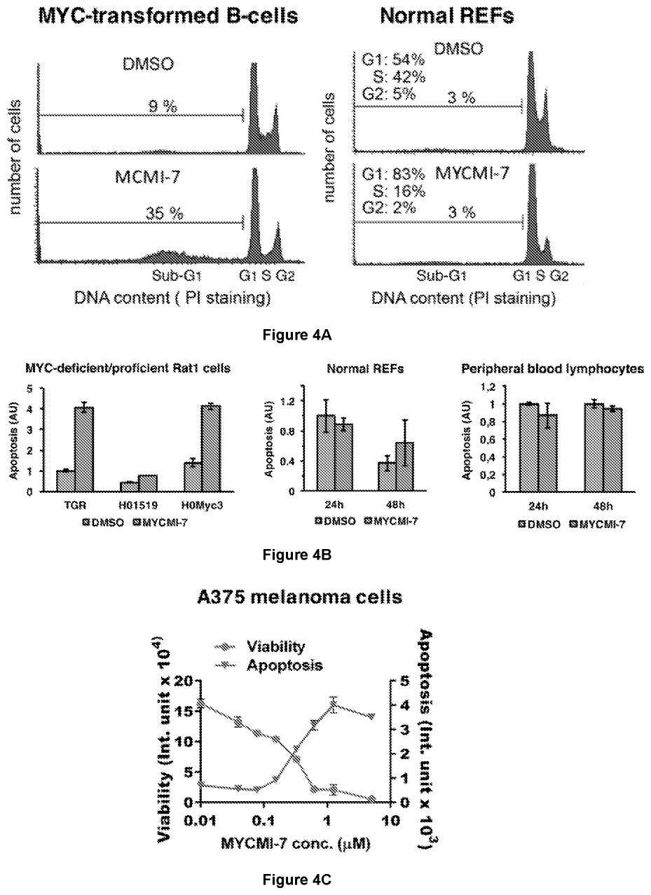

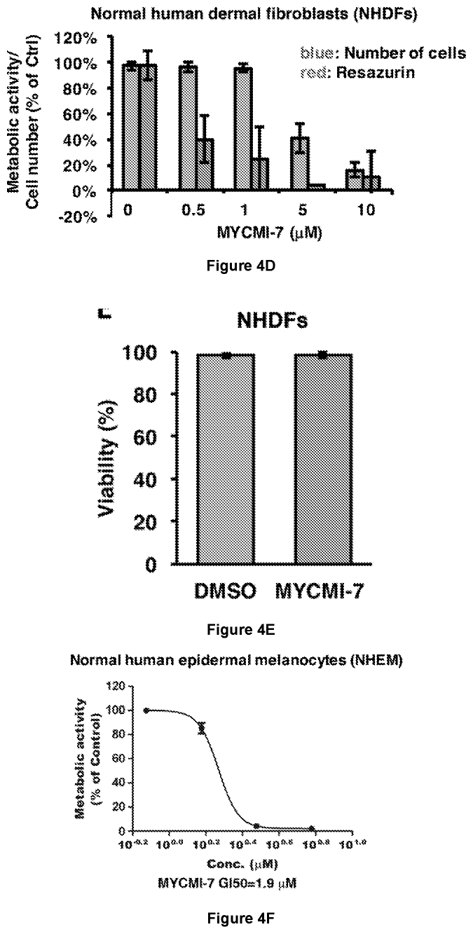

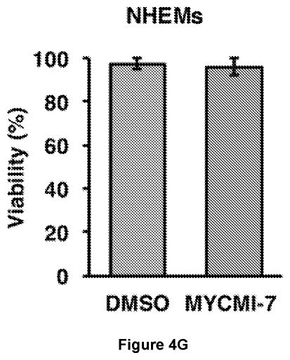

We next studied the effect of MYCMI-7 on the cell cycle utilizing P493-6 cells, which harbor a doxycycline-regulated MYC transgene (Schuhmacher M, et al. (1999) Control of cell growth by c-Myc in the absence of cell division. Curr Biol 9(21):1255-1258; the contents of which are incorporated herein by reference). Cells synchronized in G₀/G₁ were allowed to re-enter the cell cycle by doxycycline-withdrawal while treated with MYCMI-7 or DMSO. Compared to DMSO, MYCMI-7-treated cells showed a higher G1/S ratio, but also strongly induced cell death, as evidenced by the high proportion of sub-G1 cells (FIG. 4A left panel). In contrast, MYCMI-7 induced G1 arrest but no increase in cell death in normal REFs (FIG. 4A right panel). Further, MYCMI-7 induced apoptosis in immortalized TGR1 and MYC-reconstituted H015.19 (HOMyc3), but not in MYC knockout H015.19 Rat1 cells (FIG. 4B), demonstrating that MYCMI-7-induced apoptosis is MYC-dependent. Titration in A375 melanoma cells showed that MYCMI-7 reduced viability and inversely induced apoptosis in a dose-dependent manner with an GI₅₀ in the nanomolar range (FIG. 4C). Looking at different normal cells, we found that MYCMI-7 did not induce apoptosis in normal REFs or normal human peripheral blood lymphocytes (FIG. 4B). Normal human dermal fibroblasts (NHDF) treated with MYCMI-7 for 3 days showed a lower overall cell number and metabolic activity when normalized to exponentially growing DMSO-treated cultures at low micromolar concentrations, while cell viability was unaffected. This suggests that MYCMI-7 induced growth arrest without killing the cells (FIG. 4D and E). Also, in primary normal human epidermal melanocytes (NHEM), MYCMI-7 reduced metabolic activity, but did not induce cell death (FIG. 4F-G).

In conclusion, although MYCMI-7 affected growth of tumor cells and normal cells at similar concentrations, MYCMI-7 mainly triggered apoptosis in tumor cells but induced growth arrest with maintained viability in normal human and rodent cells.

Biological Example 5: MYCMI-7 does not induce DNA damage signaling at active concentrations and acts independently of topoisomerase 2 and p53 Considering the structural resemblance of MYCMI-7 to ellipticine (FIG. 1A), which has been described as a DNA intercalator and topoisomerase 2 (TOP2) inhibitor (Froelich-Ammon S J, Patchan M W, Osheroff N, & Thompson R B (1995) Topoisomerase II binds to ellipticine in the absence or presence of DNA. Characterization of enzyme-drug interactions by fluorescence spectroscopy. J Biol Chem 270(25):14998-15004; the contents of which are incorporated herein by reference), we were concerned about possible effects of MYCMI-7 on TOP2 activity and DNA damage. We first investigated whether MYCMI-7 is able to inhibit TOP2A, which is the main TOP2 isoform in cycling cells (Pommier Y, Leo E, Zhang H, & Marchand C (2010) DNA topoisomerases and their poisoning by anticancer and antibacterial drugs. Chem Biol 17(5):421-433; the contents of which are incorporated herein by reference). First, we utilized an in vitro TOP2A decatenation assay. TOP2 enzymes decatenate kinetoplast DNA (kDNA) converting a network of interlocking DNA rings into individual rings that can be separated by agarose gel electrophoresis. The TOP2 inhibitor doxorubicin (DOX) started to inhibit TOP2A activity at 3 μM with complete TOP2A inhibition at 10 μM (data not shown), while the TOP1 inhibitor camptothecin has no effect, as predicted. MYCMI-7 also exhibited TOP2A inhibitory activity at concentrations higher than 10 μM with complete TOP2A inhibition at 100 μM. This is consistent with off target effects starting to appear in MYC knockout cells at higher concentrations than 10 μM (data not shown). Ellipticine had no effect on TOP2A activity at any concentration used (data not shown). This indicated that MYCMI-7 was able to inhibit TOP2A activity in vitro above 10 μM.

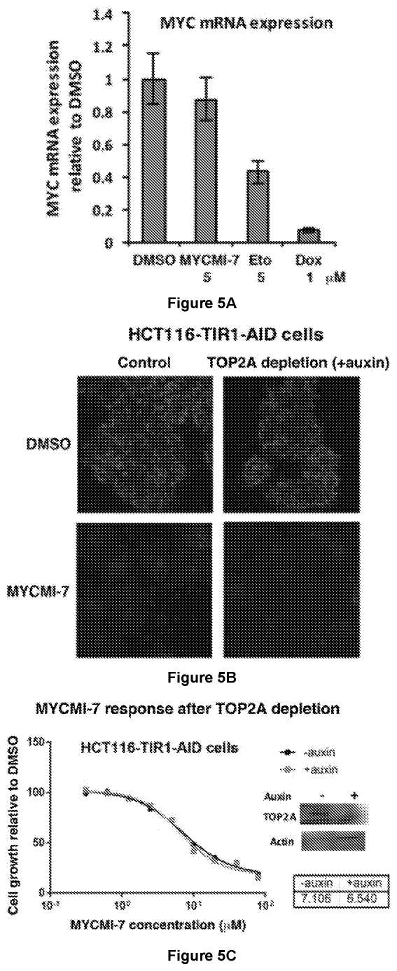

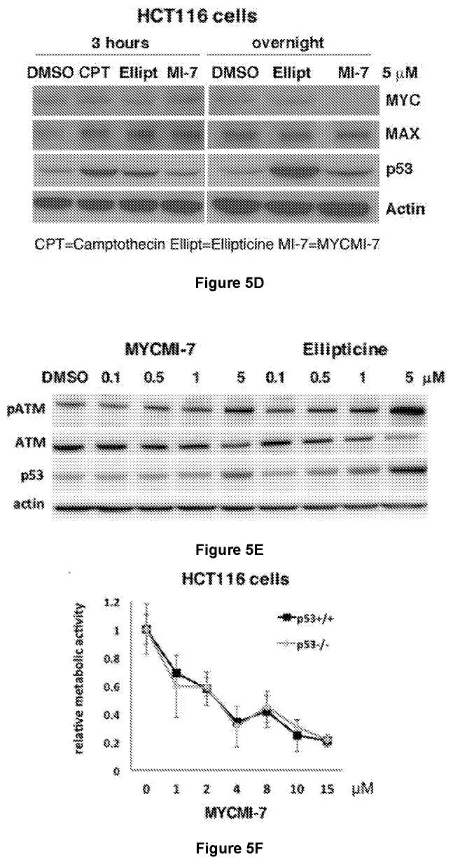

To address if the anti-proliferative effects of MYCMI-7 in cells could be explained by DNA intercalation and/or TOP2-inhibitory activity, we first compared its ability to down-regulate MYC mRNA expression, which is one well-known effect of DNA intercalators/topoisomerase inhibitors such as DOX and etoposide (ETO) (Fornari F A, Jr., et al. (1994) Induction of differentiation and growth arrest associated with nascent (nonoligosomal) DNA fragmentation and reduced c-myc expression in MCF-7 human breast tumor cells after continuous exposure to a sublethal concentration of doxorubicin. Cell Growth Differ 5(7):723-733; Horiguchi-Yamada J, et al. (2002) DNA topoisomerase II inhibitor, etoposide, induces p21WAF1/CIP1 through down-regulation of c-Myc in K562 cells. Anticancer Res 22(6C):3827-3832; the contents of each of which are incorporated herein by reference). MYC expression was strongly reduced by 1 μM DOX, and also by 5 μM ETO, while 5 μM MYCMI-7 had only minor effect on MYC expression at concentrations where it readily induced growth arrest in cells, suggesting that MYCMI-7 acts through a distinct mechanism. To investigate if TOP2A is required for MYCMI-7 activity, we made use of HCT116-TIR-AID cells with auxin-regulatable destruction of TOP2A (Yesbolatova A, et al. (2020) The auxin-inducible degron 2 technology provides sharp degradation control in yeast, mammalian cells, and mice. Nat Commun 11(1):5701; the contents of which are incorporated herein by reference). Pre-treatment with auxin to deplete TOP2A had no effect on MYCMI-7-induced inhibition of MYC:MAX interactions in the cells as measured by isPLA (FIG. 5B), nor did auxin treatment of HCT116-TIR1-AID or siRNA-mediated knockdown of TOP2A in MDA-MB-231 cells affect the growth inhibitory effect of MYCMI-7 (FIG. 5C), arguing against a mechanistic involvement of TOP2A in the action of MYCMI-7. Further, in contrast to ellipticine and the TOP1 inhibitor camptothecin, which induced p53 expression as expected, 5 μM MYCMI-7 had no effect on p53 expression early (3 hrs) and only a minor increase late (24 hrs) after treatment in HCT116 cells and in MYCN-amplified Kelly neuroblastoma cells (FIG. 5D). Moreover, while 5 μM ellipticine strongly induced phosphorylation of ATM in parallel with increased p53 expression, indicative of DNA damage response (DDR) signaling, treatment with 5 μM MYCMI-7 showed only a slight increase in these markers (FIG. 5E). To address whether p53 is required for the effects of MYCMI-7, we utilized wt and p53-deficient HCT116 cells. Titration of MYCMI-7 in these two cell lines showed no difference with respect to growth response or MYC expression (FIG. 5F)

In conclusion, although MYCMI-7 can inhibit TOP2A in vitro at higher M concentrations, there were very little signs of such effects in cells at relevant concentrations, as evidenced by the lack of DDR signaling, p53 induction or MYC mRNA reduction. Further, the anti-MYC activity of MYCMI-7 was not dependent on TOP2A or p53.

### Biological Example 6: MYCMI-7 is a Potent Inhibitor of Ex Vivo Growth of Primary Patient Derived Glioblastoma and AML Tumor Cells

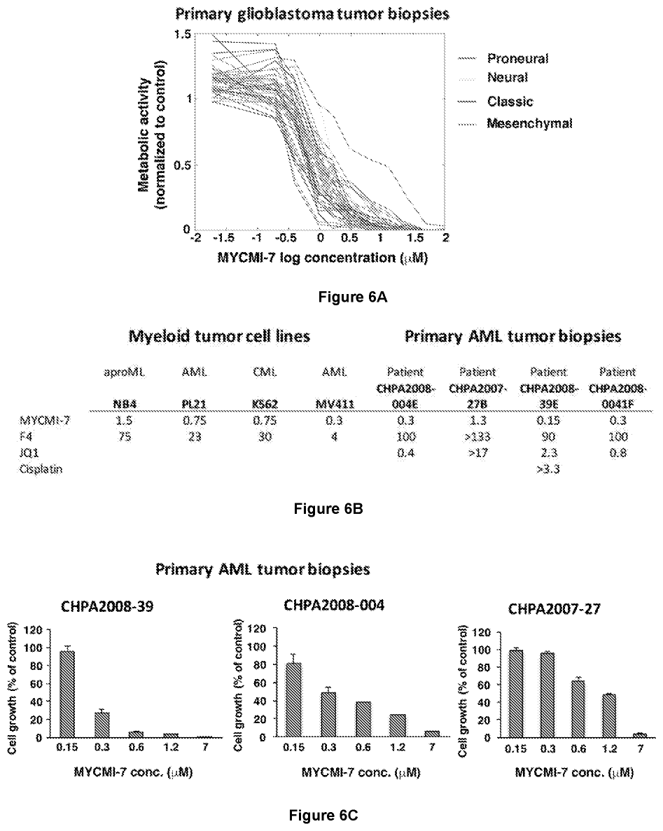

Next, we investigated the efficacy of MYCMI-7 using primary patient tumor samples in culture. An ex vivo screen of cells derived from primary glioblastoma tumor biopsies of 42 patients, representing different subtypes of glioblastoma (proneural, neural, classical and mesenchymal) (Johansson P, et al. (2020) A Patient-Derived Cell Atlas Informs Precision Targeting of Glioblastoma. Cell Rep 32(2):107897; the contents of which are incorporated herein by reference), was performed in 2D cultures. MYCMI-7 was very potent with GI₅₀ in the submicromolar range, and did not seem to discriminate between subtypes (FIG. 6A). MYCMI-7 also showed potent dose-dependent inhibition of growth of four primary patient derived AML cell cultures as well as of established AML/CML cell lines, with GI₅₀ ranging from 0.15-1.3 μM, and were in all cases more efficient than the MYC:MAX inhibitor 10058-F4, the bromodomain inhibitor JQ1, and cisplatin (the latter used in one case) (FIG. 6B and C).

### Biological Example 7: MYCMI-7 Reduces Tumor Burden in a MYC-Driven AML Mouse Model

Encouraged by the results in established tumor cell lines, primary patient-derived tumor cells and normal cells, we next decided to apply MYCMI-7 in vivo in mice. First, we performed a pharmacokinetic study of the behavior of the molecule in mice. Analysis by mass spectrometry of plasma samples collected at 1, 2, 4, and 24 hours after intraperitoneal (i.p.) injection of MYCMI-7 at a concentration of 6.25 mg/kg body weight showed an estimated half-life of 1.5 hrs for the compound in plasma (data not shown). MYCMI-7 could not be detected in the brain, but intracerebroventricular injection of 10 ml MYCMI-7 direct into the brain showed no signs of toxicity at the highest concentration (10 μM) (data not shown). In vitro analysis of MYCMI-7 ADME (absorption, distribution, metabolism, excretion) properties demonstrated that it was stable against oxidative metabolism in human and mouse liver microsomes but had strong efflux based on Caco2 cell assay (data not shown).

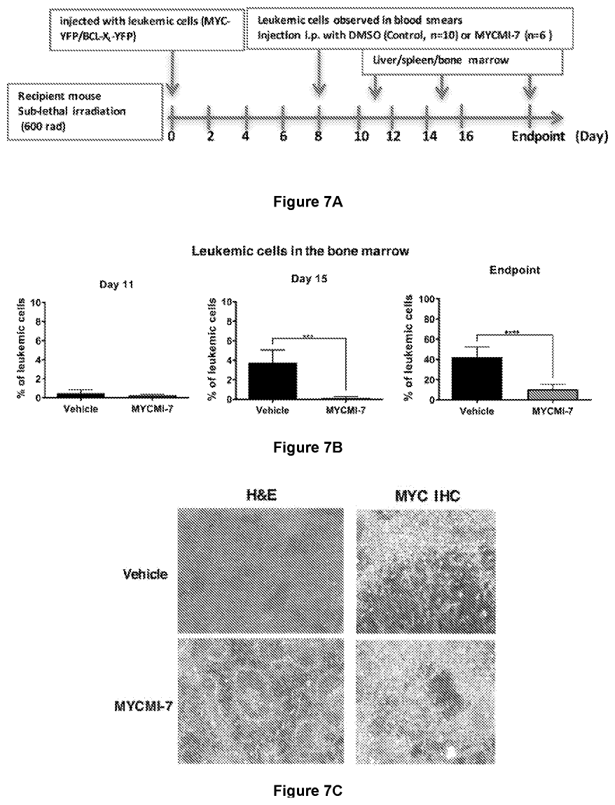

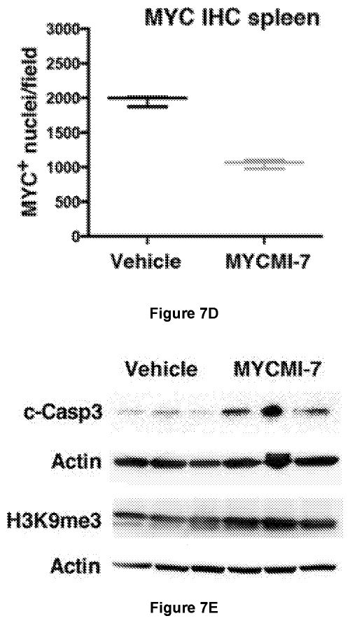

We next investigated potential anti-tumor effects of MYCMI-7 in vivo. First, we utilized a MYC/BCL-X_{L}-driven AML mouse tumor model (Bazzar W, et al. (2021) Pharmacological inactivation of CDK2 inhibits MYC/BCL-XL-driven leukemia in vivo through induction of cellular senescence. Cell Cycle 20(1):23-38; Hogstrand K, et al. (2012) Inhibition of the intrinsic but not the extrinsic apoptosis pathway accelerates and drives MYC-driven tumorigenesis towards acute myeloid leukemia. PLoS One 7(2):e31366.; the contents of each of which are incorporated herein by reference). Purified AML cells from spleens of moribund mice placed in culture were highly sensitive to MYCMI-7 treatment, with an GI₅₀ less than 1 μM (data not shown). After tail vein injection of the purified AML cells into sub-lethally irradiated syngeneic recipient mice, leukemic blasts were first observed in blood smears at day 8, at which point the mice were treated with 12.5 mg/kg MYCMI-7 daily by i.p. injection. The mice were then sacrificed according to scheme illustrated in FIG. 7A. GFP positive leukemic cells were hardly detectable by flow cytometry in the bone marrow at day 11, but reached around 4% at day 15 in vehicle-treated mice, while still undetectable in the MYCMI-7-treated mice, (FIG. 7B, left and middle panels). At the end point (20+/−4 days), bone marrow of vehicle-treated mice consisted of around 40% leukemic cells, but significantly less, around 10%, in MYCMI-7-treated mice (FIG. 7B, right panel). Similar results were obtained from the spleen (data not shown). Interestingly, spleens of MYCMI-7-treated mice retained a more normal spleen structure compared to the collapsed structure of vehicle-treated mice (FIG. 7C). Further, MYC expression was strongly reduced in MYCMI-7-compared with vehicle-treated animals in leukemic cells, as determined by immunohistochemistry (IHC), suggesting that MYCMI-7 reaches its target in vivo (FIG. 7C, D). Western blot analysis showed an increased expression of cleaved caspase 3 but also of H3K9me3 in leukemic spleens of mice treated with MYCMI-7 compared with vehicle (FIG. 7E), suggesting concurrent induction of both apoptosis and senescence. Despite this, there was no significant difference in mouse survival between the treatments (data not shown). Further, there were no signs of severe side effects of MYCMI-7 treatment; all mice retained their weight over time of the experiment (data not shown).

In conclusion, MYCMI-7 treatment delayed onset of AML, decreased tumor burden, reduced MYC expression and induced apoptosis and senescence markers in leukemic cells, but did not improve overall survival. One should, however, bear in mind that this an extremely aggressive mouse tumor model.

### Biological Example 8: MYCMI-7 Reduces Tumor Burden and Increases Survival in Xenograft Tumor Models of MYCN-Amplified Neuroblastoma and Basal-Like Breast Cancer

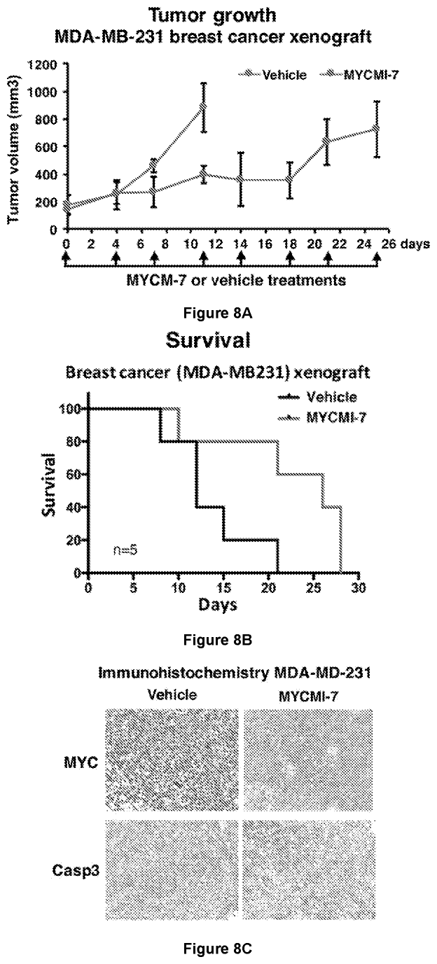

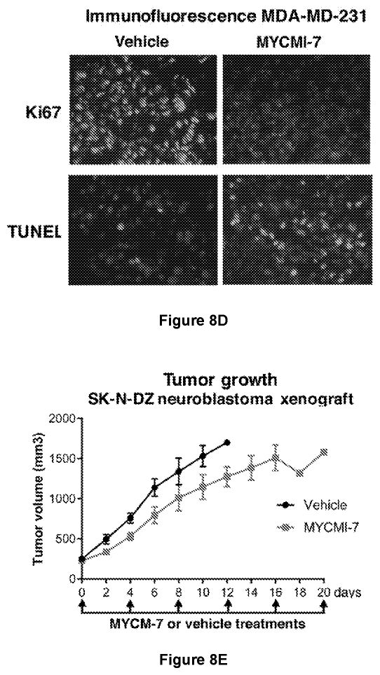

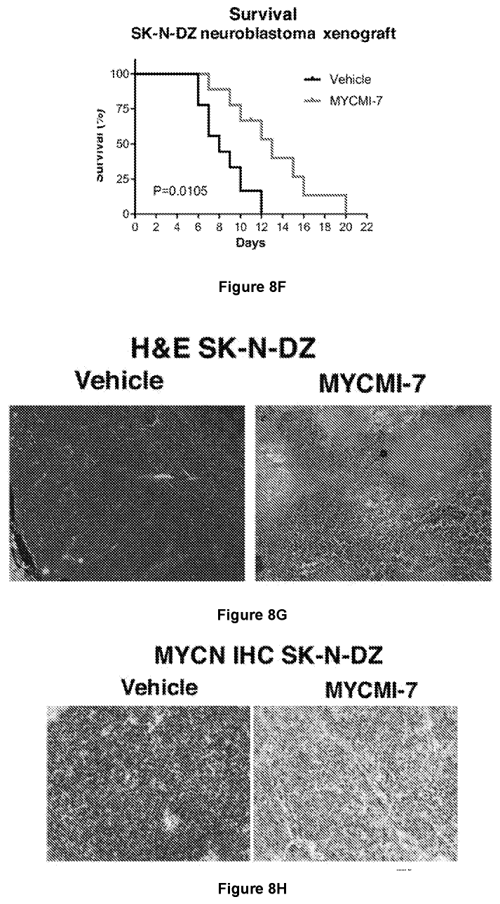

We next studied the effects of MYCMI-7 in a xenograft model of the human basal-like breast cancer cell line MDA-MB-231. The cells were highly sensitive to MYCMI-7 treatment in cell culture (data not shown), with a GI₅₀ around 1 μM. NOD/SCID mice with established MDA-MB-231 xenograft tumors were treated with 6.25 mg/kg MYCMI-7 or vehicle twice weekly by intratumoral injection. After a few days of MYCMI-7 treatment and onwards, tumor growth slowed down considerably compared with vehicle (FIG. 8A), and resulted in a significantly increased survival of the mice (FIG. 8B). H&E staining of tumor areas showed extensive necrosis/apoptosis in MYCMI-7-treated mice (data not shown). Further, IHC staining of tumors revealed reduced expression of MYC and increased caspase 3 expression in response to MYCMI-7, indicative of apoptosis induction (FIG. 8C). The latter was also supported by increased TUNEL staining (FIG. 8D). The tumors were also characterized by reduced proliferation and microvascular density as determined by immunofluorescence staining of Ki67 and CD31 (FIG. 8D), which both are typical characteristics of MYC inhibition in vivo (Castell A, et al. (2018) A selective high affinity MYC-binding compound inhibits MYC:MAX interaction and MYC-dependent tumor cell proliferation. Sci Rep 8(1):10064; Sodir N M, et al. (2011) Endogenous Myc maintains the tumor microenvironment. Genes Dev 25(9):907-916.; the contents of each of which are incorporated herein by reference).

We next evaluated the efficacy of MYCMI-7 in the MYCN-amplified SK-N-DZ neuroblastoma xenograft model in NMRI nu/nu mice. After the tumors had reached a volume of around 0.2 ml, MYCMI-7 or vehicle were administered by intratumoral injection at a dose of 6.25 mg/kg body weight twice a week. MYCMI-7 treatment reduced tumor growth compared with vehicle (FIG. 8E), resulting in a significantly increased survival (FIG. 8F). H&E staining of tumor tissue showed areas of massive apoptosis and/or necrosis as well as hemorrhage (FIG. 8G). Further, strong reduction in MYCN expression was observed in tumor cells by IHC (FIG. 8H), again indicating the MYCMI-7 had reached its target in tissues.

Taken together, these results show that MYCMI-7 has the capacity to inhibit growth of breast cancer and neuroblastoma xenograft tumors in vivo and to increase survival of mice, as well as to reduce MYC/MYCN expression and tumor cell proliferation, and to trigger apoptosis and necrosis in tumor tissues.

1. A compound of formula I: 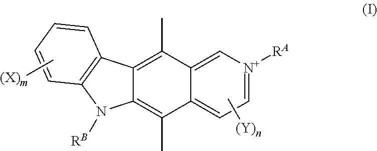
or a pharmaceutically-acceptable salt and/or detectably labelled derivative thereof for use in the treatment of a cancer,
wherein:
R^{A} represents C₁₋₆ alkyl optionally substituted by one or more group selected from F, oxo and R^{A1};
R^{A1} represents —OR^{A2}, —N(R^{A3})R^{A4}, halo, CN, or phenyl optionally substituted by one or more halo;
R^{A2} represents H or C₁₋₃ alkyl optionally substituted with one or more F;
R^{A3} and R^{A4} each represent H or C₁₋₃ alkyl optionally substituted with one or more F,
or alternatively R^{A3} and R^{A4} may be taken together to form a C₄₋₅ alkylene optionally substituted with one or more F;
R^{B} represents H or C₁₋₃ alkyl optionally substituted with one or more F;
m represents 0, 1, 2, 3 or 4;
n represents 0, 1, 2 or 3;
each X independently represents —OR^{A5}, —N(R^{A6})R^{A7}, halo, —CN or C₁₋₃ alkyl optionally substituted with one or more F; and
R^{A5} represents H or C₁₋₃ alkyl optionally substituted with one or more F;
R^{A6} and R^{A7} each represent H or C₁₋₃ alkyl optionally substituted with one or more F; and
each Y represents methyl optionally substituted with one or more F.

2. The compound for use of claim 1, wherein R^{A} represents:
C₁₋₃ alkyl optionally substituted by one or more F; or
C₁₋₃ alkylene-R^{A1} optionally substituted by one or more F.

3. The compound for use of any one of claims 1 to 2, wherein R^{A} represents:
C₁ alkyl; or
C₁₋₂ alkylene-R^{A1}.

4. The compound for use of claims 1 or 3, wherein R^{A1} represents N(R^{A3})R^{A4}.

5. The compound for use of any one of claims 1 to 4, wherein R^{A3} and R^{A4} each represent ethyl, or R^{A3} and R^{A4} are taken together to form a C₅ alkylene.

6. The compound for use of any one of claims 1 to 5, wherein each X independently represents —OR^{A5}, halo or C₁ alkyl

7. The compound for use of any one of claims 1 to 6, wherein R^{B} represents H.

8. The compound for use of any one of claims 1 to 7, wherein m represents 1 or 0.

9. The compound for use of any one of claims 1 to 8, wherein compound of formula I is a compound of formula Ib: 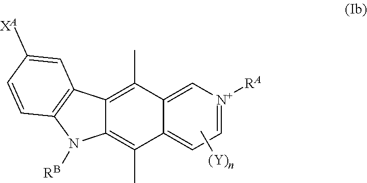
wherein R^{A}, R^{B}, m, n and Y are as defined in any one of claims 1 to 8 and X^{A} represents H or X as defined in any one of claims 1 to 8.

10. The compound for use of any one of claims 1 to 9, wherein n represents 0.

11. The compound for use of any one of claims 1 to 10, wherein the compound of formula I is as defined in any one of Examples 1 to 6.

12. The compound for use of any one of claims 1 to 11, wherein the detectably labelled derivative comprises a detectable label of formula -L-Z, wherein
L represents a direct bond or a suitable linker group; and
Z represents a detectable group.

13. The compound for use of claim 12, wherein Z is a group of formula II

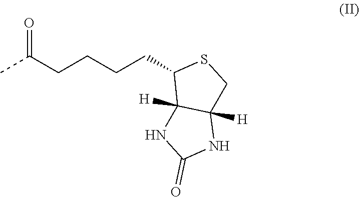

14. The compound for use of claim 12 or claim 13, wherein L is a group of formula —(R^{a0}—W¹)ᵥ—, wherein:
each R^{a10} independently represents C₁₋₆ alkylene optionally substituted with one or more R^{b10};
W¹ represents —O— or —N(R^{c10})—;
each R^{b10} represents fluoro;
each R^{c10} independently represents H or C₁₋₃ alkyl optionally substituted with one or more fluoro; and
v represents 1 to 6.

15. The compound for use of any one of claims 1 to 14, wherein the cancer is characterised by increased MYC activity.

16. The compound for use of any one of claims 1 to 15, wherein the cancer is selected from the list consisting of:
Burkitt's lymphoma;
ovarian cancer, such as ovarian cancer with BRCA alterations;
basel-like breast cancer;
esophageal squamous cell carcinoma;
colon cancer;
endometrial cancer;
neuroblastoma;
small cell lung carcinoma;
medulloblastoma, such as group 3;
pancreatic cancer;
head and neck cancer;
prostate cancer; and
hepatocellular carcinomas.

17. A pharmaceutical composition comprising a compound of formula I as defined in any one of claims 1 to 14, or a pharmaceutically acceptable salt and/or detectably labelled thereof, and optionally one or more pharmaceutically-acceptable excipient.

18. A pharmaceutical composition as defined in claim 17 for use in the treatment of a cancer, such as a cancer as defined in any one of claims 15 or 16.

19. A combination product comprising:
(I) a compound of formula I as claimed in any one of claims 1 to 14, or a pharmaceutically acceptable salt and/or detectably labelled derivative thereof,
and
(II) one or more other therapeutic agent that is useful in the treatment of cancer,
wherein each of components (I) and (II) is formulated in admixture, optionally with one or more a pharmaceutically-acceptable excipient.

20. A kit-of-parts comprising:
(a) a pharmaceutical composition as defined in claim 17,
and
(b) one or more other therapeutic agent that is useful in the treatment of cancer, optionally in admixture with one or more pharmaceutically-acceptable excipient,
which components (a) and (b) are each provided in a form that is suitable for administration in conjunction with the other.

21. A compound of formula I: 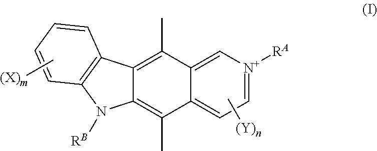
or a pharmaceutically-acceptable salt and/or detectably labelled derivative thereof, for use as a pharmaceutical,
wherein:
wherein R^{A}, R^{B}, X, Y, n and m are as defined in any one of claims 1 to 14.

22. A compound of formula I: 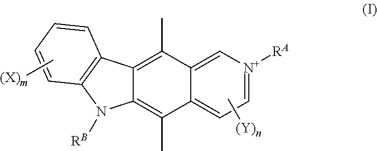
or a pharmaceutically-acceptable salt and/or detectably labelled derivative thereof,
wherein:
wherein R^{A}, R^{B}, X, Y, n and m are as defined in any one of claims 1 to 14,
with the proviso that the compound of formula I is not
(a) as depicted in Compound Example 1 (i.e. 2-(5,11-dimethyl-6H-25-pyrido[4,3-b]carbazol-2-yl)-N,N-diethylethanamine); or
(b) as depicted in any one of Compound Examples 2 to 6.


SEQUENCE LISTING (12 sequences)

SEQ ID NO: 1 - DNA, 19 bp, Homo sapiens
  source 1..19: mol_type = genomic DNA
acatcgctcagacaccatg

SEQ ID NO: 2 - DNA, 22 bp, Homo sapiens
  source 1..22: mol_type = genomic DNA
tgtagttgaggtcaatgaaggg

SEQ ID NO: 3 - DNA, 20 bp, Homo sapiens
  source 1..20: mol_type = genomic DNA
tctgcttgatattggcggtg

SEQ ID NO: 4 - DNA, 22 bp, Homo sapiens
  source 1..22: mol_type = genomic DNA
ggctcagctatgattctcactc

SEQ ID NO: 5 - DNA, 20 bp, Homo sapiens
  source 1..20: mol_type = genomic DNA
gggttttcttggctctcgtc

SEQ ID NO: 6 - DNA, 21 bp, Homo sapiens
  source 1..21: mol_type = genomic DNA
ccttatcacggtaccaacagc

SEQ ID NO: 7 - DNA, 21 bp, Homo sapiens
  source 1..21: mol_type = genomic DNA
ctgcgatactgggagtactgg

SEQ ID NO: 8 - DNA, 18 bp, Homo sapiens
  source 1..18: mol_type = genomic DNA
ccaccccagcacatcttc

SEQ ID NO: 9 - DNA, 21 bp, Homo sapiens
  source 1..21: mol_type = genomic DNA
tggctggtgactatgagtttg

SEQ ID NO: 10 - DNA, 20 bp, Homo sapiens
  source 1..20: mol_type = genomic DNA
cagcattgccagaaatccac

SEQ ID NO: 11 - DNA, 20 bp, Homo sapiens
  source 1..20: mol_type = genomic DNA
aggtgaccccaagaaaatgg

SEQ ID NO: 12 - DNA, 21 bp, Homo sapiens
  source 1..21: mol_type = genomic DNA
agccttcttgcctttcttctg